(19) United States
(12) Patent Application Publication
(10) Pub. No.: US 2012/0115147 A1
(43) Pub. Date: May 10, 2012

(54) NEUROPSYCHIATRIC TEST REPORTS

(76) Inventor: Jay L. Lombard, New City, NY (US)

(21) Appl. No.: 13/290,603

(22) Filed: Nov. 7, 2011

Related U.S. Application Data
(60) Provisional application No. 61/410,523, filed on Nov. 5, 2010.
(60) Provisional application No. 61/528,583, filed on Aug. 29, 2011.

(51) Int. Cl. G06Q 50/22; C12Q 1/68

(52) U.S. Cl. 435/6.11; 705/2

(57) ABSTRACT

Methods and reports for presenting genetic information that is patient-specific and relevant to treatment of neuropsychiatric disorders, including treatment resistant depression. The methods and reports described include genotype information for each of six specific genetic loci and allow patient-specific therapy for the effective treatment of treatment resistant disorders (TRD).

### CROSS REFERENCE TO RELATED APPLICATIONS

This patent application claims priority to U.S. Provisional Patent Application No. 61/410,523, titled “TREATMENT RESISTANT DEPRESSION DIAGNOSTIC TEST REPORT” and filed on Nov. 5, 2010. This patent application also claims priority to U.S. Provisional Patent Application No. 61/528,583, titled “INTERPRETIVE BIOMARKER SCREENING REPORTS FOR DIAGNOSIS AND TREATMENT OF PSYCHIATRIC DISORDERS” filed on Aug. 29, 2011.

This patent application may be related to any of the following: U.S. patent application Ser. No. 12/790,262, titled “METHOD FOR ASSESSMENT AND TREATMENT OF DEPRESSION VIA UTILIZATION OF SINGLE NUCLEOTIDE POLYMORPHISMS ANALYSIS” and filed on May 28, 2010; U.S. patent application Ser. No. 13/074,967, titled “METHODS FOR ASSESSMENT AND TREATMENT OF MOOD DISORDERS VIA SINGLE NUCLEOTIDE POLYMORPHISMS ANALYSIS” and filed on Mar. 29, 2011; U.S. patent application Ser. No. 13/177,032, titled “APOE4 AND APOJ BIOMARKER-BASED PREVENTION AND TREATMENT OF DEMENTIA” and filed on Jul. 6, 2011; and U.S. patent application Ser. No. 13/210,808, titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS” filed on Aug. 16, 2011. Each of these patent applications is herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

This patent application relates to personalized diagnostic reports. In particular, this application describes integrated personalized diagnostic reports configured to provide streamlined information to guide a physician treating a psychiatric patient wherein the information presented is specific to the particular patient.

### BACKGROUND OF THE INVENTION

Virtually all brain disorders may cause psychiatric symptoms; thus, the term “neuropsychiatric disorders” may refer to brain disease or dysfunction that causes psychiatric symptoms. Examples of neuropsychiatric disorders include neurodegenerative diseases (Alzheimer's disease, frontotemporal lobar degeneration, depression (including treatment resistant depression, bipolar depression, etc.), PTSD and other anxiety disorders, autism, ADHD, traumatic brain injury, metabolic encephalopathy, etc.

In the last fifty years, a tremendous amount of research has begun to elucidate the causes, characteristics and treatments of many neuropsychiatric disorders. Unfortunately, this this process has not efficiently translated into effective patient treatments, in part because this information has proven difficult to interpret and apply to patient care. Further, neuropsychiatric disorders are notoriously difficult to diagnose because existing categories of disorders are imprecise; there is a great deal of overlap in the symptoms of these conditions. Thus, it is difficult to accurately categorize patients. In general, categorical tests for neuropsychiatric disorders have not proven effective in accurately diagnosing and treating patients, as there is a great deal of variation in patient outcomes between patients categorized with the same diagnosis.

Although various research and clinical studies have looked for diagnostic and therapeutic indicators in an almost overwhelming variety of genetic markers, markers of gene expression and markers of protein expression, this vast and growing body of data has proven difficult to interpret. Most physicians are unable synthesize the tremendous amount of information on possible risk factors and indicators in order to apply this information clinically to diagnose and/or treat patients. Thus, there is an as-yet unmet need for reports, panels and/or kits that would allow a medical professional to apply the most relevant genetic, epigenetic and proteomic tests in a meaningful manner. It is also critical to provide tests that allow the medical profession to understand and interpret the results of such tests.

Described herein are systems and kits, including panels, assays and articles of manufacture, including reports, which meet this need by providing interpretive and directed reports, particularly for the treatment of neuropsychiatric disorders such as depression. Because of the confusing and contradictory information available for even those genes established as implicated for treatment of depression and dementia, it would be beneficial to provide systems that (1) select the relevant genes or genetic markers, epigenetic markers and/or protein/expression markers; (2) provides information or links to the key information such as the relevance and meaning of each indicator or screen member: (3) suggests or provide relevant therapies based on the results of these tests; and/or (4) provide an indication of the confidence/reliability of the interpretive information provided. In particular, described herein are methods and articles of manufacture that may provide a concise reporting to a medical professional to help make diagnostic and/or therapeutic decisions.

The interpretive reports described herein may also be useful in developing and understanding new sites of psychotropic drugs, as well as a previously undisclosed explanation on how single nucleotide polymorphisms (SNPs) in certain genes are related to subtypes of depression and their relative response to different classes of agents. There is a growing need to provide an interpretation of information provided by genetic testing (particularly multiple genetic tests) to the clinician or learned intermediary to aid in treatment and/or diagnosis. The articles of manufacture described herein may include interpretive logic configured to analyze the results of all of the SNP assays and to provide interpretive comments, wherein the interpretive logic is encoded for processing on a processor or any other easily accessed and reviewable form. The interpretive comments may indicate the effect of any identified SNPs on the regulation of neurotransmitter activity, ionic channel function and metabolism. This is an important element of our discovery because an educational system is a critical requiem to understand the interpretation of genes properly as well as to be in compliance with regulatory guidelines. Unfortunately, without providing a proper context, genetic test results can lead to confusion rather than clarification in a clinical setting. In subsequent paragraphs, particular language of interpretation for various biomarker test results will be provided. Within these descriptions, clarification regarding both the potential benefits and limitations of biomarker analysis is provided, as well as recommended preferred therapeutic interventions based upon patient genotype.

Genes associated with neurotransmitter, ionic channels (calcium, sodium and potassium) and metabolic pathways have been found to be abnormal in patients with various neuropsychiatric disorders. For instance, genes which regulate serotonin pathways, including genes coding for receptors, metabolism and reuptake mechanisms, are associated with depression. Furthermore, other genetic-neurotransmitter pathways, including dopamine, norepinephrine and glutamate may be associated with depression or risk of dementia. Regarding ion channels, pathological states in the brain can result from changes in non-mutated channels which alter membrane excitability. Genes related to cerebral metabolism, such as methylation and the like, also impart changes with neuropsychiatric implications. Lastly, genes which regulate immune processes are also relevant in clinical assessments as variants in glial cell activity have been associated with depression, schizophrenia, bipolar disease and dementia.

Unfortunately, the heterogenous nature of gene findings in these disorders suggests that neuropsychiatric disorders themselves are heterogenous and require a dimensional, rather than categorical approach. By analyzing disorders using a spectrum of biomarkers, including single nucleotide polymorphism based gene analysis, subtypes of neuropsychiatric conditions can be differentiated and treated in a personalized manner. This analysis allows a deeper understanding of a patient's health across a variety of neuropsychiatric categories. Further, the employment of such analysis will allow mental health professionals to treat individuals with more specific and targeted interventions. The approach described herein may therefore be used to reveal gene influences on trait components of a variety of neuropsychiatric disorders (regardless of categorical classification) and, may help identify subpopulations of patients that can benefit from more targeted pharmacotherapy.

As an example, a single nucleotide polymorphism in the gene that regulates dopamine can be associated with reduced levels of this neurotransmitter with parallel changes in an individual's behavior. Patients with a dopamine based SNP differ not only in their symptoms but their response to therapies as well; however, such patients may be otherwise hard to identify, based solely on their behavior. In addition, by examining a variety of biomarkers a deeper understanding of a patient's treatment needs may be achieved. For example, a mood complaint such as depression can either be a consequence of a genetic defect that effects serotonin metabolism, but also can be a consequence of a genetic polymorphism or genetic defect in a gene that regulates dopamine, or some other neurotransmitter. As a similar example, depression can be etiologically associated with a SNP in glutamate in one individual, and with a SNP related to dopamine or norepinephrine in another. Thus, it would be beneficial to provide method and articles (including systems, reports, kits and the like) that are capable of conveniently and simultaneously informing a physician of a variety of relevant factors informing patient care.

The recognition of the distinction in the genetic and biochemical heterogeneity related to the expression of subtypes of depression has important therapeutic implications. Frequently, an individual with a mood disturbance does not respond favorably to a specific first class of therapeutic agents but may respond to a different second class of therapeutic agents. As an example, an individual who is experiencing depression due to a specific SNP related dopamine metabolism defect will not respond or will respond less favorably to a serotonin modulating agent. In clinical practice, this can happen when a psychiatrist treats a patient with depression who possesses a SNP associated with a dopamine related defect with a serotonin modulating drug like sertraline or paroxetine instead of a dopamine modulating drug such as buproprion. In these instances, the drug may produce a worsening of symptoms instead of improving them.

Conversely, an individual with a SNP associated with serotonin metabolism will respond less favorably to a dopamine modulating agent. Frequently in such patients, depressive symptoms will not improve or may in fact, worsen. Unfortunately, psychiatrists currently administer medications for depression solely on a trial and error basis. The lack of diagnostic specificity frequently leads to ineffective treatments or a delay in the proper treatment.

Thus, a common problem in the management of mood disorders is a lack of diagnostic specificity and/or treatments which are not coupled to the unique neurotransmitter disturbance related to depression. Provided herein is a method of using the analysis of biomarkers for an individual related to neurotransmitter function as an aid to diagnosis and choice of therapeutic treatment.

It is further an object of this description to set forth the specific functional axes related to both excitatory and inhibitory pathways and anatomical regions in the brain that are causally associated with the biochemical and neurochemical abnormalities associated with various neuropsychiatric conditions. These axes each have associated biomarkers which may be probed. The ability to accurately identify variations of functionally related biomarkers as taught herein represents an important advance in the field of mental health.

Lab diagnostics in CNS disorders often lack specificity and sensitivity. A simple, but novel solution is to recognize that an integrated approach to the diagnosis of these disorders, rather than a single lab modality, is required. Thus, while there may be limitations to diagnosing a disorder based upon the utilization of genetic based technology exclusively, the application of an analysis of biomarker signals integrated under a broader diagnostic framework which includes one or more of genetic analysis, epigenetic analysis, methylation determination, proteomics, and the like, will increase the confidence of the diagnostic signal and lead to previously unrealizable treatment efficacy and specificity.

For example, it has long been suspected that in schizophrenia a cluster of genes is likely to contribute a gene dosing effect. However, even when detecting these genes, it is unclear whether any of these genes are actually expressed. Thus, it is not sufficient to know that a patient has a genetic polymorphism linked to an increase in risk; there is a requirement in the field to develop a more holistic analysis which should also include, in addition to risk genes, the actual detection of altered gene expression. Gene expression, in addition to gene inheritance may provide a more reliable use of biomarkers in neuropsychiatry.

Expression may be based upon unique transcriptional analysis and post translational modifications, including epigenetics, e.g., methylation of specific cpg islands, and/or protein expression. Several examples will be set forth below related to specific disorders but the teaching can be more generally applied to other conditions not mentioned.

Thus, in some embodiments, the approach outlined is a teaching which requires an analysis at multiple levels of molecular biology: genes, methylation and or histone modifications of such genes, proteomics and the like.

Depression, and particularly treatment resistant depression, is one area where there is pressing need to provide a biomarker-based test and report that collects relevant biomarkers and presents the results of testing these biomarkers to a physician in an interpreted manner. Between 5-10% of adults worldwide suffer from depression, and the economic costs to society and the personal costs to individuals and families, associated with depression are enormous. Within a 15-month period after having been diagnosed with depression, sufferers are four times more likely to die as those who do not have depression. Almost 60% of suicides have their roots in major depression, and 15% of those admitted to a psychiatric hospital for depression eventually kill themselves. In the U.S. alone, the estimated economic costs for depression in 1990 exceeded $44 billion. The World Health Organization estimates that major depression is the fourth most important cause worldwide of loss in disability-adjusted life years, and will be the second most important cause by 2020.

A variety of pharmacologic agents are available for the treatment of depression. Significant success has been achieved through the use of serotonin reuptake inhibitors (SRIs), norepinephrine reuptake inhibitors (NERIs), combined serotonin-norepinephrine reuptake inhibitors (SNRIs), monoamine oxidase inhibitors (MAOIs), glutamate inhibitors or other compounds. However, even with these options available, many patients fail to respond, or respond only partially to treatment. Additionally, many of these agents show delayed onset of activity, so that patients are required to undergo treatment for weeks or months before receiving benefits. Most currently available antidepressants take 2-3 weeks or more to elicit a response.

Traditional therapies can also have significant side effects. For example, more than a third of patients taking SRIs experience sexual dysfunction. Other problematic side effects include gastrointestinal disturbances, often manifested as nausea and occasional vomiting, agitation, insomnia, weight gain, onset of diabetes,

Patients that fail to respond to these standard/traditional depression therapies may be classed as suffering from treatment resistant depression (TRD, also referred to as refractory depression or treatment refractory depression). TRD is often described as depression that does not respond to different antidepressant medications from more than at least two different classes, or different treatments.

In the clinic, 40-50% of depressed patients who are initially prescribed antidepressant therapy do not experience a timely remission of depression symptoms. This group typifies treatment-refractory depression, that is, a failure to demonstrate an “adequate” response to an “adequate” treatment trial (that is, sufficient intensity of treatment for sufficient duration)). Moreover, about 20-30% of depressed patients remain partially or totally resistant to pharmacological treatment.

There is increasing evidence implicating the role of neurotransmitters in depression, in particular the monoamines serotonin, noradrenaline, dopamine, as well as the excitatory amino acid glutamate. Many of the tricylic antidepressants (TCAs), selective serotonin re-uptake inhibitors (SSRIs) and monoamine oxidase inhibitors (MAOIs) effective in the treatment of depression increase the availability of the catecholamines (noradrenaline and dopamine) and indolamines (serotonin) in the central nervous system (CNS). The clinical efficacy of these agents has given rise to the catecholamine-indolamine hypothesis of depression. This theory postulates that a certain level of amines and/or receptor sensitivity to catecholamines functions to generate a normal mood. Receptor insensitivity, a depletion of monoamines, or a decrease in their release, synthesis or storage has been postulated to lead to depression.

Personalized medicine is considered a young but rapidly advancing field of healthcare that is informed by each person's unique clinical, genetic, genomic, and environmental information. Because these factors are different for every person, the nature of diseases—including their onset, their course, and how they might respond to drugs or other interventions—is as individual as the people who have them.

The goal of personalized medicine is to customize or individualize treatments based on the particular genetic, genomic, and clinical information specific to a patient, thereby allowing accurate predictions to be made about a person's susceptibility of developing disease, the course of disease, and its response to treatment.

In order for personalized medicine to be used effectively by healthcare providers and their patients, these findings must be translated into precise diagnostic tests and targeted therapies. This has begun to happen in certain areas, such as testing patients genetically to determine their likelihood of having a serious adverse reaction to various cancer drugs. Recently, work has begun to extend this testing to drugs used in other fields, including psychopharmacology.

The complete sequencing of the human genome provided a first step towards understanding the biological workings behind countless medical conditions. Although the field of personalized medicine is advancing at a fast pace, as new disorders are linked to particular genetic predispositions and mutations, adoption of such personalized markers for disorders and treatments has been slowed by the overwhelming amount of information available.

Although personalized medicine offers patients and clinicians numerous advantages, there are also increasing risks arising from the narrow focus and resulting myopia when examining complex disorders in light of only a few genetic linkages. Medical practitioners are often caught between having too little information or too much information. If a medical practitioner examines only some of the genes which may be implicated in a disorder, he or she may miss essential information. Alternatively, providing information about too many genes and genetic variations may prevent a concise diagnosis, resulting in confusion.

These problems are particularly present in the area of personalized medicine for use in treating psychological disorders such as depression. Although numerous genetic loci have been implicated as contributing to both the development of depression and the response of a patient to treatments for depression (see, e.g., U.S. patent application Ser. No. 12/790,262, previously incorporated by reference), it has proven difficult to determine which genetic factors are of significance in treating the patient. This patent application describes the heterogenous nature of depression, and in particular some of the genotypes and phenotypes that may be correlated to depression, which may be based upon neurotransmitter subclassifications.

In particular, when prescribing treatment for treatment resistant depression (TRD), there is a strong need to provide a means for reducing the overwhelming amount of genetic data available into a reduced and simplified format to guide a medical practitioner in treating TRD.

### SUMMARY OF THE INVENTION

Described herein are methods and systems, including articles of manufacture such as reports, for guiding therapeutic treatment of neuropsychiatric disorders. For example, described herein are systems, methods and articles of manufacture relevant to the treatment of depression and particularly treatment-resistant depression (TRD).

In general, the systems described herein may provide a dimensional, rather than categorical, assay, screen, test, or report relevant to treating a neuropsychiatric disorder. The dimensional assays, and dimensional assay reports, described herein typically use a collection or set of biomarkers that are relevant to one or more areas useful for understanding excitatory and inhibitory systems of the brain underlying many classes of neuropsychiatric disorder. These areas may be defined or described based on the anatomical and/or functional biological relationships, which may also correlate with the primary neurotransmitter pathways for that area. Of particular interest are three areas, which are described in greater detail below: the autonomic arousal area (or axis), the emotional valence and reward and executive brain function area (axis), and the memory and cognition area (which may also be referred to as the cognition, memory, excitatory neurotransmission, long term potentiation area) (axis).

The autonomic arousal area (or axis) functionally relates to stress and autonomic hyperactivity. The functional brain circuits involved typically include the amygdala and hypothalamus. The principle neurotransmitter pathways implicated for this area include the serotonin/norepinephrine neurotransmitter pathways. Dysregulation of these neurotransmitter receptors, particularly in these brain regions, may result in problems of the autonomic arousal axis. Representative gene markers may include, but not limited to: serotonin transporter s/s or s/Lg (also restrictive sequence 25531), FKBP5 (e.g., rs 3800373), 5HT1a (e.g., c1019g), Ace (e.g., insertion/deletion rs 4291), Neuropeptide Y (e.g., rs 16147), COMT (e.g., 158 val/met). In some variations a potential treatments for this endogenotype may involve noradrenergic modulators, angiotensin receptor blockers or other agents which can reduce autonomic hyperactivity.

The emotional valence and reward/executive brain function area (axis) relates to the pain/pleasure response may involve the functional brain circuits of the pre-frontal cortex, ventral striatum, and nucleus accumbens regions of the brain. The principle neurotransmitter pathway implicated in this axis is dopamine (dopaminergic); dysregulation of dopamine neutotransmission in these regions may result in dysfunction of this axis. Representative gene markers may include, but not limited to: DRD2 (e.g., rs 1076560, 2734839, taqa1 deletion, rs 1800497, 141 ins/del), SNAp25 (rs 363039, 3746544, 1051312), SLC6A3 (e.g., rs 37020, DAT1 9 repeat VNTR, 10 repeat VNTR), COMT (e.g., 158 val/met), MAOA (e.g., uVNTR), DBH (e.g., 1021). Potential treatment for dopamine hypo-expression genes based on the biomarkers examined may include stimulants, Buproprione, Seligiline, S adenosylmethionine, and/or COMT inhibitors. Potential treatment for dopamine hyper-expression based on the biomarkers examined may include antipsychotics.

Cognition, memory, excitatory neurotransmission, long term potentiation axis may include the functional brain circuits in the hippocampus, including the neurotransmitter systems such as the glutamate neurotransmitter pathway, calcium channels, sodium channels, and the like. Representative gene markers in this axis include: CACNA1C (e.g., rs 1006737, 2370419), SCN1A (e.g., rs 3812718), SLC1A1 (glutamate transporter) (e.g., rs 10491734, 10491733, 4740788), ANK3 (e.g., rs 10994336), GRIK4 (e.g., rs 12800734). Potential treatments for genetic evidence of excessive excitatory neurotransmission in this axis may include: lithium, Lamotrogine, Valproic Acid, Dextromethorphan, Nimodipine and other calcium channel blockers, memantine, magnesium. Furthermore, the choice whether to use a calcium channel based mood stabilizer or sodium channel modulator may be assisted by an analysis of these variants. Ankyrin, the gene encoded by ANK3, is enriched at the nodes of ranvier and mediates the aggregate activity of sodium channels in these axonal pathways. Variants of ANK3 may selectively respond therefore to sodium channel inhibitors such as lamotrogine.

Information, and particularly biomarker information, for a particular individual in any, or preferably all, of these areas may be helpful. Thus any of the systems (including the methods and reports) described herein may include at least one biomarker for a particular group, or multiple biomarkers for each group. A three-axis group may also include information on at least one biomarker for each of these pharmacodynamic areas just described.

In some variations a system, method of article of manufacture may include a fourth axis related to metabolism. This axis may be a pharmacokinetic axis, relevant to metabolism (including drug metabolism). For example, a metabolism area (axis) may include one or more biomarkers for cytochrome p450 mediated hepatic degradation related to pharmokinetics, methylation, neuroimmune function, blood brain barrier status, brain lipid signaling and insulin pathways. For example representative gene markers may include, but not limited to: 2D6, 2C19, 3A4, ABCB1 (e.g., rs 1045642, 1128503), 5HT2C (e.g., 759 c/t), MTHF, MCR4 (e.g., rs 2229616), IDE (e.g., rs 65838, 7910972).

Thus, in one variation a method, system or article of manufacture may feature at least one biomarker from each of these four axes: the autonomic arousal axis, the emotional valence and reward and executive brain function axis, the memory and cognition axis, and the metabolism axis. The clusters of genetic biomarkers related to each (or all) of these axes can be used both for clinical and research purposes. In use, panels indicating the results of biomarkers from these axes can provide a significant amount of information to alert the clinician to a potential abnormality in a prominent neurotransmitter pathway. The pathways implicated in these axes mediate and form the biological basis of behavior, including assessment of external risk and fear (autonomic arousal axis, axis I), novelty seeking, motivation and evaluation of significance (emotional valence and reward and executive brain function axis, axis II), and cognitive processes including memory and long term potentiation (the memory and cognition axis, axis III).

Currently, the majority of agents used to treat neuropsychiatric disorders relate to the modulation of serotonin, norepinephrine, dopamine and glutamate; genetic biomarkers associated with these pathways can therefore be employed for treatment decision processes. It may be of particular use to include pharmacodynamic biomarkers along with pharmacokinetic biomarkers. In addition to specific genes related to the metabolism of drugs, the identification of genetic variants related to insulin or lipid metabolism and the like, may lead to the employment of novel therapeutic interventions which are not typically classified as being directly psychotropic. These may include, for example, the use of PPAR agonists in bipolar disease, Methylfolate for depression, and other typically off-label applications of drugs, based on the results of the biomarker assay as interpreted by the systems and methods described herein.

The three pharmacodynamic assays described above may have parallel critical brain regions and their corresponding neurotransmitter pathways.

In some variations, the dimensional assay should include at least one marker from each of the three axes described (and in some instances, a fourth from the fourth, metabolic, axis). These markers do not, by themselves, indicate a particular diagnosis for a neuropsychiatric disorder (e.g., they are not traditional categorical or “diagnostic” markers), but may cut across different categories of neuropsychiatric disorders. These areas provide dimensional and phenomenological data about inherited predispositions and vulnerability to pathological states, and may thus provide clinical information useful to treat a variety of neuropsychiatric disorders.

As mentioned above, in some variations the methods, kits and reports described herein provide an integrated analysis of a set of biomarkers, such as genetic markers, epigenetic markers and/or protein expression markers. The set of biomarkers may be specifically selected to optimize the therapeutic information provided, as described in greater detail herein. The application and incorporation of such a methodology will enhance diagnostic certainty where analysis of any of these markers separately and in isolation provides only limited insight.

One variation of such a method includes a process for biomarker detection (e.g., gene detection such as single nucleotide polymorphisms, copy number variation and the like). Biomarker detection may be determined on one more appropriate platforms. Various platforms may include, but are not limited to, the Affymetric gene chip, Taqman or Illumina and the like, and validation via PCR. Development of these chips can be further validated by robust multi chip analysis algorithms and subjected to ontological analysis by a variety of different bio informatics tools. Following detection, a report may be generated, including a description of the physiological significance of the results of the biomarker test, and additional interpretive information. The interpretive information provided may include a score or weighting index indicating a confidence level for the interpretive information. This score or index may indicate the number of studies supporting the interpretive comment, the size of the studies supporting the interpretive comment and/or the existence of any disputing or contradictory studies. Some variations, references or links to references may also be provided. Both the testing and the report may be configured to extract patient information most relevant to treatment.

Several genome-wide association studies (GWAS) have suggested that the combination of several genes, analyzed in a specific cluster, may account for various psychiatric and neurological disorder phenotypes. The observations of such studies are likely incomplete, because they fail to take into account altered protein expression and epigenetic factors. For example, the serum proteome has high complexity with thousands of non-redundant proteins due to multiple post translational modifications.

Thus, an additional step in the methods and systems for examining and reporting on clinically relevant biomarkers in neuropsychiatry may include an examination of RNA expression. RNA expression analysis may further refine diagnostic specificity as it relates to the actual encoding of DNA in regions of particular interest. Suitable modalities to include are an expressed sequence tag analysis.

Another step may include an analysis of the actual protein expression of an altered gene through proteomics. Therefore, one or more proteomic based technologies may be incorporated into the integrative platform described herein. For example, subtractive proteomics, which compares two or more proteomes to identify proteins that are specifically enriched or depleted, may be used as one peptide substrate mapping strategy. Isotope affinity tags are another suitable method of protein detection which may be used.

In some variations DNA methylation analysis may be incorporated into the systems, methods and reports described herein. The methylation status of CpG islands may correlate with the activity of transcribed genes, which are generally unmethylated. Technologies to assess DNA methylation, including bisulfite reactions are typically hampered by variability, but may benefit from the combined and tiered approach described herein. PCR-based assays and other improvements to the state of the art may also be incorporated into the method, systems, kits and reports described below.

The human genome can be methylated in regions called cpG islands, which control gene transcription through the methylation of methyl-CpG binding domains. When methylated, gene inactivation occurs due to chromatin condensation. In some variations, this epigenetic indicator (e.g., methylation of one or more region of DNA) may be detected and interpreted. Methylation detection methods may determine methylation patterns in a particular genomic locus. Various methods can be employed to detect methylation of these genes which is well known to those skilled in the art. For example, Bisulfite methylation test may be used or oligonucleotide reiteration test may be used.

Methylation detection may be a useful tool as a neuropsychiatric biomarker in the systems, reports and methods described herein for: (1) predicting drug response by measuring gene inactivation in responders to non-responders in a region of interest; and (2) analyzing markers in disease detection. For example, methylation detection has proven helpful for treatment of lupus patients by observing hypomethylation of DNA in the circulation of these patients. Similarly, the methylation status of GSTP1 is being explored as a marker for prostate or colorectal cancer. Methylation is also being explored as an indicator of drug response. For example, in breast cancer low methylation of PITX2 in lymph nodes may predict recurrence after Tamoxifen treatment and in glioblastoma, MGMT methylation may predict response to alkylating agents. In psychiatry and neurology, examples of methylation of particular genes may be also important, including analysis of BDNF, serotonin transporters and the like. As mentioned above, we herein predict and propose that the combination of an epigenetic indicator such as methylation in conjunction with genetic markers in the locus identified herein and/or protein expression makers may prove substantially more powerful and reliable. Thus, an integrative biomarker assay could include specific analysis of gene methylation patterns in critical brain pathways such as those described herein.

In general, any of the genes described herein may be used as biomarkers by testing for polymorphisms, mutations, insertions, deletions, translocations, methylation, histone deacetylation, etc. The proteins expressed by any of these genes may also be tested for expression level, localization, folding (or miss-folding), and the like.

The methylation status, including hypo- and hyper-methylation of certain genes may be a marker of neuropsychiatric disorder. In general, epigenetic modulations may play an important role in fine-tuning of gene expression in response to environmental factors. For instance, using quantitative methylation specific PCR, MB-COMT promoter has been seen to be hypo-methylated in DNA derived from the saliva in schizophrenia compared to control subjects, suggesting that DNA methylation analysis of MB-COMT promoter in saliva can potentially be used as an epigenetic biomarker for disease state. Further, the CpG at T102C of the HTR2A polymorphic site and neighboring CpGs were approximately 70% methylated both in the patients and controls. qMSP analysis revealed that the cytosine of the T102C polymorphic site was significantly hypo-methylated in SCZ compared to the controls. Thus, for example, Cytosine methylation of HTR2A at T102C polymorphic site in DNA derived from the saliva can potentially be used as a diagnostic, prognostic, and/or therapeutic biomarker in SCZ and BD.

As another example, the methylation status of retinoic acid-related orphan receptor alpha (RORA) has been implicated in Autism. Methylation of RORA was confirmed by bisulfite sequencing and methylation-specific PCR; this data has revealed decreased expression of RORA proteins in the autistic brain.

Methylation may also be used as a biomarker for depression and post-traumatic stress disorder (PTSD). For example, SLC6A4 methylation levels appear to modify the effect of the number of traumatic events on PTSD after controlling for SLC6A4 genotype. Persons with more traumatic events were at increased risk for PTSD, but only at lower methylation levels. At higher methylation levels, individuals with more traumatic events were protected from this disorder. Depressive symptoms were more common among those with elevated buccal cell 5HTT methylation who carried 5HTTLPR short-allele. Thus hypomethylation of SLC6A4 may be used as a marker of depression and/or PTSD.

Protein expression may also be used as a biomarker. The examination of protein expression, including proteomics, may use an analytic method such as mass spectrometry and the like. Protein expression may also be examined by immunological methods (e.g., immunocytochemical detection). An abnormal protein may correspond to an abnormal biological state, whereas a gene abnormality is more trait dependent. Proteins that are found to be more prevalent in diseased patient samples compared to normals may be an important potential disease biomarker for disorders like dementia, schizophrenia, autism, head injury and the like. However, a search for any particular biomarkers in disease-free or asymptomatic individuals is neither cost effective nor efficient. Therefore, it may be significantly more effective to combine an assessment of genetic risk and/or epigenetic risk with a proteomic analysis.

It should be noted that there currently exist commercial assays which are used for psychiatric diagnosis. A clear distinction between the systems, reports and methods described herein and these other tests includes the difference between analyzing pharmacodynamic (PD) genes and pharmacokinetic (PK) genes. In the latter example, PK genes provide information related to drug metabolism but do not provide any insight into trait dependent and specific neurochemical factors related to neuropsychiatric conditions. These trait-dependent factors, which are components of the current disclosure, include assessment of stress resilience, risk of neurodegeneration associated with a co morbid mood disorder, risk of psychiatric decompensation or cyclical mood disturbances, subendophenotypes of depression, and the like. The systems described herein may examine biomarkers indicative of pharmacodynamic (PD) traits. These biomarkers test for the activity or interactions of one or more members of a biological pathway, including those pathways involved in neurotransmission. For example, the methods described herein may examine genes related to neurochemical imbalances. Such tests may be broadly applied to the genes involved in at least the following pathways: Serotonin, dopamine, norepinephrine, glutamate and the hypothalamic pituitary adrenal axis. Additional gene analysis also relates to calcium channels, sodium channels, potassium channels which are also relevant to neuropsychiatric disorders and response to particular interventions. Other genes of importance relate to metabolism. These genes include brain glucose utilization, methylation, inflammation and the like.

Specific genes within these areas are described in the paragraphs herein but are not limited to this disclosure. Thus, while the present invention describes polymorphisms in specific serotonin pathways, it is recognized that other polymorphisms in the serotonin pathway are contemplated as within the scope of this disclosure. Similarly, biomarkers (such as snps) in the glutamate, dopamine, norephinephrine, and hypothalamic pituitary adrenal axis may be examined as well.

Gene detection such as single nucleotide polymorphisms, copy number variation and the like on various platforms known to those skilled in the art, such as the Affymetric gene chip, Taqman or Illumina and validation via PCR, can be utilized. Several methods have been advanced as suitable means for detecting the presence of low levels of a target nucleic acid in a test sample. One category of such methods is generally referred to as target amplification, which generates multiple copies of the target sequence, and these copies are then subject to further analysis, such as by gel electrophoresis, for example.

There are many variations of target nucleic acid amplification, including, for example, polymerase chain reaction (PCR), which has been disclosed in numerous publications. The most commonly used target amplification method is the polymerase chain reaction (PCR), which consists of repeated cycles of DNA polymerase-generated primer extension reactions. Each reaction cycle includes heat denaturation of the target nucleic acid; hybridization to the target nucleic acid of two oligonucleotide primers, which bracket the target sequence on opposite strands of the target that is to be amplified; and extension of the oligonucleotide primers by a nucleotide polymerase to produce multiple, double-stranded copies of the target sequence. The discovery of thermostable nucleic acid modifying enzymes has contributed to rapid advances in nucleic acid amplification technology.

The exemplary systems, screens and methods described herein may include assays for determining genetic indicators (including genetic polymorphisms), epigenetic markers (such as methylation status), and protein expression. Such assays may include known tests, assays or methods, which may be integrated or combined in known or novel ways. For example, diagnostic kits using “gene chip” technology may be used to determine genetic and/or epigenetic information about particular genes of interest, and may be integrated with protein indicators including immunosassays or the like. Examples of these are provided herein.

For example, in some variations a neuropsychiatrically specific oriented kit may be provided. This kit (which may include a gene chip) may be built to provide a practical and clinically relevant tool in practice in neuropsychiatry because it is able to translate GWAS level research findings into a clinically practical framework. In this fashion, the benefit of focusing on a narrow and pre-selected group of SNPs relates to the application and context of the results in an integrative clinical setting.

As a specific example of the methods of diagnosing and/or treating a neuropsychiatric diagnosis described above, the inventor has applied one variation to identify and propose treatment of a previously unrecognized mood disorder. For example, the inventor has discovered that individuals with a COMT val/val polymorphism in epistasis with MTHF TT may display a phenotype characterized by a subcortical type of mood disorder. These individuals commonly are abulic, dysthymic, and anergic. This phenotype may be expressed secondary to reduced prefrontal dopamine as a consequence of these genes being in epistasis, resulting in excess dopamine degradation. Thus, a system, report or method may examine the combination of COMT and MTHF and/or dopamine neurotransmitter pathway genes. One or more genetic markers, epigenetic markers and/or protein expression may be examined to determine if a patient has or is at risk for the correlated abulic, dysthymic, and anergic phenotype.

In another example, the combination of serotonin short alleles and CACNA1C variants has also been linked by the inventor to a particular phenotype which may be specifically amenable to treatment, either to enhance treatment or to select between available treatments that would otherwise be seemly equivalent based only on the phenotype presented to the physician. For example, SSRI induced mania may be higher in these patients. Other general and specific examples are illustrated below.

For example, described herein are methods of presenting patient-specific pharmacodynamics information relevant to the treatment of a neuropsychiatric disorder. These methods may be used to improve psychiatric diagnosis, including depression, bipolar, schizophrenia, dementia and the like. In general, any of the methods, systems, and articles of manufacture described herein may use a specified cluster of biomarkers. As described herein, these clusters of biomarkers may include, for example, a representative set of biomarkers having particular relevance across a cross-section neurotransmitter pathways, neurofunctional pathways, and/or neuroanatomical pathways. These biomarkers may be derived from the compactification and compression method described herein. For example, the biomarkers may include one or more biomarkers from each of the patient's autonomic arousal system, the patient's emotional valence, attention, reward and executive brain functions, and the patient's cognition and memory. Biomarkers examined may include single nucleotide polymorphisms, deletions, methylation and protein expression, and the like. The selection of biomarkers, particulary those described in greater detail below, may indicate that status or functionality of neurotransmitter pathways, the patient's neuroimmune system and/or neuroendocrine system. The methods described herein may provide an integrative framework applied to these biomarkers in which component elements are interpreted in a holistic neural net framework, rather than reductionist fashion.

Further, any of the methods described herein may incorporate specific brain imaging modalities, including magnetic resonance spectrocopy and the like. The incorporation of these biomarkers and their interpretation in clinical practice is also described. In particular, the devices, systems and methods described herein allow interpretation of key sub-sets of biomarkers which address the translation of research findings into clinically meaningful data. For example, the systems, methods and reports described herein provide both raw biomarker test results for a specific and meaningful group of biomarkers, as well as interpretive data including clinical and research findings specific to the patient's biomarker test results. In some variations this interpretive data is ranked, weighted or indexed to provide a confidence level to the physician or medical professional. Thus, the methods, devices and systems described herein may provide clinical support which includes specific educational material for patients and/or clinicians.

Further, the methods, devices and systems described herein may provide analytical methods to enhance the signal to noise ratio related to the use of biomarkers in psychiatry.

In some variations, the methods of presenting patient-specific pharmacodynamics information relevant to the treatment of a neuropsychiatric disorder include the steps of: providing a patient identifier; presenting a description of a biomarker test result specific to the patient; presenting an interpretive analysis of the neurophysiological significance of the biomarker test result for the patient, wherein the interpretive analysis comprises pharmacodynamics information; and presenting a weighted index of confidence level for the interpretive analysis.

These methods may be improve treatment, and in some variations may also be used to help identify and/or diagnose patients. In some variations, the methods may be used to help delineate specific treatment interventions based upon the results of the biomarkers.

The step of providing a patient identifier may include generating a report including any patient identifying mark, code, name, symbol, or the like. For example, the patient identifier may include a patient number or patient name. The patient name may be kept confidential in some variations. In variations in which the method includes providing a copy of the results, the results copy may include a written patient identifier as part of the copy of the results.

The step of presenting a description of a biomarker test result specific to the patient may include a listing or output of the raw result of the biomarker test and/or an amended form of the results. For example, when snp biomarkers are used, the presence or absence of the snp tested may be provided. In some variations the raw biomarker test result is not provided, but only a summary of the result is included (e.g., “the patient tested positive for . . . ” a particular biomarker). In some variations, the test results may indicate a polymorphism, deletion, repetition, insertion, methylation, expression level, expression localization, activity, or metabolites of one or more gene, protein, or neurotransmitter.

In general, any of the steps of presenting information (e.g., presenting a description, presenting an interpretive analysis, presenting a weighted index, etc.) may include generating a report including the presented information, or including the presented information on a single report. As discussed herein, the report may be a single page or multiple pages.

The step of presenting an interpretive analysis of the neurophysiological significance of the biomarker test result for the patient, wherein the interpretive analysis comprises pharmacodynamics information, may include providing any appropriate type of interpretive analysis and comments. Appropriate interpretive analysis typically includes a description of the physiological significance of the biomarker test result. For example, the interpretive analysis may indicate associations with neuropsychological disorders, drug response, patient behaviors, treatment outcomes, or the like in patients with the same biomarker test results. The interpretive analysis may also include a description of the gene and/or protein, and/or biological pathway associated with the particular biomarker. In some variations the interpretive analysis may also include association studies, such as gene response association studies, describing or summarizing research and/or clinical studies on the biomarker and any associations based on the presence and/or absence of the biomarker.

In some variations the interpretive analysis may also include a visual representation of a region of the patient's brain affected by the underlying biomarker (e.g., the gene and/or protein being tested by the biomarker test). The visual representation may be generic (e.g., not taken from the actual patient's brain). Multiple visual representations (including alternative views, color views, animations, etc.) may be provided. The interpretive analysis may also include possible drug responses.

The results may be provided in hard copy (e.g., written form) or they may be electronic, including delivered as a web page, PDF, or other “virtual” document.

The step of presenting a weighted index of confidence level for the interpretive analysis may include indicating for all or some of the interpretive analysis an approximation of the confidence level for that particular portion of the interpretive analysis. For example, an index may include a “score” based on the reproducibility (or lack of reproducibility), the number of patients/subject's examined in the academic or clinical literature or references, the length of time studied, or the like. In general, these confidence level scores may be summarized in the report in a key, or they may be self-qualifying (e.g., the index may indicate “high,” “medium” or “low” confidence values). In some variations the weighted index of confidence level may include alphanumerically indexing all or a portion of the interpretive analysis with a score indicating the type and/or number of studies supporting the interpretive analysis.

Any neuropsychiatric disorder may be addressed by the methods, devices and articles of manufacture described herein, and particularly depression. For example, the neuropsychiatric disorder examined may be selected from the group including: treatment resistant depression, bipolar depression, anxiety disorders, dementia, autism, and ADHD. In some variations the patient may not be diagnosed with a particular neuropsychiatric disorder; in some variations the methods, systems and reports described herein may be used as an aid in diagnosing the patient.

Although the general method of presenting patient-specific pharmacodynamics information relevant to the treatment of a neuropsychiatric disorder includes only a single biomarker test result, it is of particular interest to examine and present patient-specific information about a set of biomarkers. In particular a set of biomarkers that include one or more markers from a subset of “axes” such as the patient's autonomic arousal system, the patient's emotional valence, attention, reward and executive brain functions, and the patient's cognition and memory. Each of these axes describes pharmacodynamic biomarkers; in some variations it may also be helpful to include one or more markers of pharmacokinetic biomarkers. Examples of specific markers are provided herein. In particular, depression (and treatment-resistant depression especially) may include one or more markers from each of patient's autonomic arousal system, the patient's emotional valence, attention, reward and executive brain functions, and the patient's cognition and memory. When examining other neuropsychiatric disorders, only one or two of these axes may be used, or entirely other axes may be chosen.

As mentioned, in some variations the biomarker provides information about the autonomic arousal system of the patient's brain. For example, the biomarker may be a marker of a gene, or a protein encoded or modulated by gene selected from the group consisting of: SERT, SLC6A4 (SERT), 5HT1a, ACE, NPY, FKBP5, and other genes associated with heightened amygdala function. In some variations a biomarker provides information about the emotional valence, attention, reward and executive brain functions of the patient. For example, the biomarker may be a marker of a gene, or a protein encoded or modulated by gene selected from the group consisting of: COMT, sigma receptors, SNAp25, MAO A, SLC6A3, DRD2, DBH. In some variations, the biomarker provides information about the patient's cognition and memory. For example, the biomarker may be a marker of a gene, or a protein encoded or modulated by gene selected from the group consisting of: CACNA1C, GRIK, GRM3, SLC1A1, ANK3, BDNF.

Also described are methods of presenting patient-specific pharmacodynamics information relevant to the treatment of a neuropsychiatric disorder including the steps of: presenting a description of a biomarker test result specific to a patient for at least one biomarker related to each of: the patient's autonomic arousal system, the patient's emotional valence, attention, reward and executive brain functions, and the patient's cognition and memory; and presenting an interpretive analysis of the neurophysiological significance of each biomarker test result for the patient, wherein the interpretive analysis comprises pharmacodynamics information. All of the variations and additional steps described above may also be applied to these methods.

Also described herein are methods of presenting patient-specific pharmacodynamics information relevant to the treatment of a neuropsychiatric disorder, the method comprising: providing a patient identifier; presenting a description of a plurality of biomarker test results specific to the patient; presenting an interpretive analysis of the neurophysiological significance of each of the biomarker test result for the patient, wherein the interpretive analysis comprises pharmacodynamics information; and presenting a visual representation of a brain region affected by each biomarker.

Articles of manufacture for assisting in the treatment of neuropsychiatric disorders are also described herein. For example, described herein are articles of manufacture comprising an interpretive neuropsychiatric report of patient-specific pharmacodynamics information relevant to the treatment of a neuropsychiatric disorder, the article of manufacture comprising: a report including a patient identifier; a description of a biomarker test result specific to the patient; an interpretive analysis of the neurophysiological significance of the biomarker test result for the patient, wherein the interpretive analysis comprises pharmacodynamics information; and a weighted index of confidence level for the interpretive analysis. The report is generally written, and the tangible medium of the report may be hardcopy (e.g., paper) or electronic (e.g., a digital file describing the written results). Thus, in any of the articles of manufacture described, the patient identifier and descriptions of the biomarker test results and interpretive analysis may be non-transiently formed on the report. The report may also be stored in any appropriate electronic medium (e.g., digital medium).

In some variations, the article of manufacture includes a plurality of descriptions of biomarker test results specific to the patient for a plurality of biomarkers, and may also include interpretive analyses of the neurophysiological significance of each of the biomarker test results for the patient.

As mentioned above, the interpretive analysis may further comprises a description of the physiological significance of the biomarker test result for the patient, a description of published studies describing similar biomarker test results, an indicator of possible drug responses, and/or a visual representation of a brain region affected by the biomarker.

In addition to the pharmacodynamics biomarker(s), the article of manufacture may also include a description of a biomarker test results for a pharmacokinetic biomarker.

The weighted index of confidence level may include an alphanumerical index of all or a portion of the interpretive analysis with a score indicating the type and/or number of studies supporting the interpretive analysis. The article of manufacture may also include a list of references specific to the patient's biomarker test result (the references may be part of the interpretive analysis).

As mentioned above, the biomarker test result may indicate a polymorphism, deletion, repetition, insertion, methylation, expression level, expression localization, activity, or metabolites of one or more gene, protein, or neurotransmitter. For example, an article of manufacture may include a test result and interpretive comments for a biomarker related to the autonomic arousal system of the patient's brain, such as a gene, or a protein encoded or modulated by gene selected from the group consisting of: SERT, SLC6A4 (SERT), 5HT1a, ACE, NPY, FKBP5, HTR1A. An article of manufacture may include a test result and interpretive comments for a biomarker related to the emotional valence, attention, reward and executive brain functions of the patient, such as a gene, or a protein encoded or modulated by gene selected from the group consisting of: COMT, sigma receptors, SNAp25, MAO A, SLC6A3, DRD2, MTHFR, DBH. An article of manufacture may include a test result and interpretive comments for a biomarker related to the patient's cognition and memory, such as a gene, or a protein encoded or modulated by gene selected from the group consisting of: CACNA1C, SCN1A, GRIK, GRM3, GRIK4, SLC1A1, ANK3, BDNF.

In some variations of the articles of manufacture described herein, the article so manufacture include an interpretive neuropsychiatric report of patient-specific pharmacodynamics information relevant to the treatment of depression, the article of manufacture comprising: a report including a description of a biomarker test result specific to a patient for at least one biomarker related to each of: the patient's autonomic arousal system, the patient's emotional valence, attention, reward and executive brain functions, and the patient's cognition and memory; and an interpretive analysis of the neurophysiological significance of each biomarker test result for the patient, wherein the interpretive analysis comprises pharmacodynamics information. The article of manufacture may also include a weighted index of confidence level for all or part of each interpretive analysis.

In some variations of the articles of manufacture described herein, the articles include an interpretive neuropsychiatric report of patient-specific pharmacodynamics information relevant to the treatment of a neuropsychiatric disorder, the article of manufacture comprising a report including: a patient identifier; a description of a plurality of biomarker test results specific to the patient; an interpretive analysis of the neurophysiological significance of each of the biomarker test result for the patient, wherein the interpretive analysis comprises pharmacodynamics information; and a visual representation of a brain region affected by each biomarker.

Also described herein are methods of diagnosing a neuropsychiatric disorder based on patient-specific pharmacodynamics information. For example, the methods may include the steps of: sampling a patient; testing the sample for at least one biomarker related to each of: the patient's autonomic arousal system, the patient's emotional valence, attention, reward and executive brain functions, and the patient's cognition and memory; providing a report including the results of the biomarker test, an interpretive analysis of the neurophysiological significance of each biomarker test result, and a weighted index of confidence level for the interpretive analysis.

Systems for performing the methods described herein are also included, as are systems for generating the articles of manufacture (e.g., reports) mentioned above. For example, a system for generating a patient-specific pharmacodynamics report relevant to the treatment of a neuropsychiatric disorder may include: an input module configured to receive at least one biomarker test result specific to a patient for each of: the patient's autonomic arousal system, the patient's emotional valence, attention, reward and executive brain functions, and the patient's cognition and memory; an analysis module coupled to the input module and configured to generate an interpretive report from the plurality of biomarker test results, wherein the analysis module generates interpretive comment for each biomarker based on the test result.

Also described herein are systems for diagnosing or guiding a therapeutic treatment of a neuropsychiatric disorder comprising: an assay for determining the status of at least one biomarker related to each of: the patient's autonomic arousal system, the patient's emotional valence, attention, reward and executive brain functions, and the patient's cognition and memory; and a report including the status of the biomarkers determined, an interpretive analysis of the neurophysiological significance of each biomarker's status, and a weighted index of confidence level for the interpretive analysis.

Also described herein are methods for simplifying and presenting patient-specific treatment information for the treatment of a psychiatric disorder, as well as customized reports presenting information to guide treatment of psychiatric disorders. In particular, described herein are reports and methods for presenting information on the treatment of treatment resistant depression (TRD) based on patient-specific information.

In general, the methods of presenting information and the presentations (e.g., reports) described herein include the presentation of patient-specific data from a core set of genetic loci which the inventors have found to be critical to guiding the treatment of depression and particularly TRD. Thus, the presentation provides epistatic information related to the core areas, axes, or loci discussed above. The axes (loci) may be referred to functionally (e.g., cognition and memory, etc.), neuroanatomically (e.g., hippocampal, limbic, etc.) or based on their principle neurotransmitter pathway (dopaminergic, gutamatergic, etc.).

For example, described herein is a method of presenting patient-specific treatment information for treatment resistant depression may include: presenting the patient-specific information for each of the core genetic loci in an epistatic group, and presenting interpretive comments for each the results. As just mentioned, the genetic loci forming a core epistatic group typically relate to genes/proteins having a functional relationship for a particular neurotransmitter pathway, and/or neuroanatomical location, and/or neurological function.

The methods and reports described herein may present the biomarker results for a patient (e.g., a patient genotype) in a single report including biomarker information from each or the four axes identified (or a subset of them), and also present interpretive comments based on the results. The interpretive comments may describe a likely drug response based on the outcome of the biomarker results. For example, a report may provide the genotypes for biomarkers of a particular epistatic loci, and may describe putative or definite links between the results of one or more biomarker and an expected clinical significance. The interpretive comments may describe the function of a particular gene generally, and may specifically describe the significance of the genetic result of the biomarker test for that gene (or all relevant outcomes/genotypes). For example, in relation to the SERT biomarker analysis: “patients with the S/S genotype do not respond as well to SSRI antidepressants and may experience more side effects,” and/or “In SSRI non-responders who exhibit the S/S allele, consideration should be given to use of a non-SSRI.”

In some variations, the systems and methods may provide a summary of the results.

Also described herein are articles of manufacture based on the concepts taught herein. One particular article of manufacture contemplated herein is a written or displayed report describing a relevant set of biomarkers, the results of the biomarker tests, and interpretive comments including in some variations genetic information that is patient-specific and relevant to treatment of a neuropsychiatric disorder. In some variations the report includes a section providing the patient's genotype. The report may also include interpretive comments for each of the axes tested. Finally, the report may include a weighting index that provides a confidence level for all or some of the interpretive comments.

In some variation the report is an electronic report. In other variations the report is a written report. The report may be coded to indicate the presence of a genetic polymorphism in each member of the core epistatic group. The report may also include a summary (e.g., a table, chart, etc.) that lists and summarizes the genotype test results; this summary may be on the first page or the top/front of the report.

The interpretive comments may be included for each biomarker examined after a description of the genotype result for that member. In general, interpretive comments may include treatment recommendations, references to scientific literature, and any other statement describing the significance of one or more genotype. Interpretive comments may provide interpretation of the significance of each of the genotypes. Interpretive comments may also provide interpretation of the significance of combinations of genotypes for different biomarkers tested, particularly those within the different axes. Interpretive comments may also provide information on the significance of particular patient phenotypes in combination with specific (including patient-specific) genotypes.

In some variations, the interpretive comments include a visual representation of the effected brain region, including a representational image of the neuroanatomical region affected by a polymorphism identified by a biomarker, for example.

Interpretive comments may be tailored to correspond to the biomarker result for an individual; in some variations, the interpretive comments are generically provided regardless of the biomarker result. In variations in which all of the possible interpretive comments are provided regardless of the biomarker results, interpretive comments that are relevant to the identified biomarker result may be highlighted.

In general, the report may highlight or separate out the biomarker results, particularly when the biomarker indicates the presence of a polymorphism or risk factor having therapeutic consequences. For example, in some variations results indicating polymorphisms may be highlighted. Highlighting may include presenting the text in a different font, color, point, or the like, including (but not limited to) boxing the text, indenting the text, boding the text, italicizing the text, underlining the text, or the like.

### BRIEF DESCRIPTION OF THE DRAWINGS

FIGS. 1A-1D show pages 1-4 of a first exemplary report.

FIGS. 2A-2D show pages 1-4 of a second exemplary report.

FIGS. 3A-3J illustrate another example of an exemplary report as described.

FIG. 4 shows one example of a visual portion of an exemplary report.

### DETAILED DESCRIPTION OF THE INVENTION

In general, the methods and reports described herein provide a clear effective means for presenting neuropsychiatric information that is patient-specific and relevant to treatment of a neuropsychiatric disorder such as depression (e.g., treatment resistant depression, bipolar depression), anxiety disorders, autism, and ADHD. By identifying and presenting a specific subset of information that is substantially relevant to the effective understanding and thus treatment of the neuropsychiatric disorder, the systems, methods and articles of manufacture described herein may enhance patient care and simplify treatment. These systems, methods and articles of manufacture may serve as tools to aid the medical professional in predicting patient response to therapies and may also be used to help guide a patient treatment regime.

Generically, the articles of manufacture described may include a report specific to a particular patient, that includes at least one (and preferably more than one) biomarker test result, and interpretive comments describing the physiological significance of the biomarker result. The report may also indicate some index or weighting of the interpretive comments. A scale or metric may be provided to understand the index or weighting. This index or weighting may reflect the confidence of the scientific data supporting the interpretive comments. For example, the index may indicate the number of studies supporting the interpretive comment, the size of the study or studies, and/or the existence of any conflicting data. The interpretive comments may also include one or more visual images representing a brain region effected or implicated by the biomarker being tested or the result of the biomarker tested.

The assays described herein may be referred to as dimensional assays, because they present dimensional, rather than categorical, information. Each dimension of the dimensional assay may describe a particular functional, anatomical and/or neurotransmitter pathway or area. These various areas may be referred to as axes. A dimensional assay may therefore include one or more (and preferably three or four axes). For example, in some variations there are three pharmodynamic areas or axes of the dimensional assay for assessment of neuropsychiatric disorders: autonomic arousal; executive brain function and emotional valence; and memory and cognition. These have parallel critical brain regions and their corresponding neurotransmitter pathways. For example, the executive brain function and emotional valence axis includes the executive region, including the frontal lobes, which are primarily regulated by dopamine, and more specifically by D1 receptors. The emotional valence region consists primarily of subcortical pathways such as the ventral striatum and the nucleus accumbens, which are also regulated by another subset of dopamine receptors (e.g., D2 receptors) and is involved in emotional valence and reward.

The autonomic arousal axis typically relates to the anterior cingulate cortex with the frontal and subcortical limbic circuitry including the amygdala, which is densely innervated by 5Ht1 a serotonin receptors and norepinephrine pathways. The function of this pathway is primarily to modulate and act as an intermediary between internal emotional states and their cognitive interpretation.

The memory and cognition axis relates primarily to the hippocampus, and is involved in cognition, memory, excitatory neurotransmission, and long term potentiation (LTP). This region of the brain is subserved by glutamate and its relationship to NMDA and AMPA receptors and their corresponding ionic channels.

In addition to these three axes, a fourth, pharmacokinetic axis may also be included. The pharmacokinetic axis typically describes various types of metabolic (metabolism) pathways, including the cytochrome p450 mediated hepatic degradation related to pharmokinetics, methylation, neuroimmune function, blood brain barrier, brain lipid signaling and insulin pathways.

A dimensional assay may include at least one marker from each of these four axes. These biomarkers do not, by themselves, typically indicate a particular diagnosis for a neuropsychiatric disorder (e.g., they are not categorical or “diagnostic” markers), but may cut across different categories of neuropsychiatric disorders. As mentioned, these areas provide dimensional and phenomenological data about inherited predispositions and vulnerability to pathological states, and may thus provide clinical information useful to treat a variety of neuropsychiatric disorders.

### Autonomic Hyperarousal Axis

The autonomic hyperarousal axis may include the primarily serotonin neurotransmitter pathways of the anterior cingulate cortex, frontal and subcortical limbic regions (including the amygdala). Recent understandings of the biological bases of depression vulnerability have revealed that both the short allele of the serotonin transporter-linked polymorphic region (5-HTTLPR) and activity in the amygdala are associated with depression. Other studies have reported amygdala hyperactivity associated with the 5-HTTLPR short allele, linking the genetic and neuroimaging lines of research and suggesting a mechanism whereby the short allele confers depression risk. Thus, biomarkers implicated in autonomic hyperarousal and biomarkers implicated in modulation of the noradrenergic (fight/flight) response are included in this axis. For example, any of the following genes may be used as biomarkers for this axis: SERT (biomarkers may include, for example, snps or epigenetic regulation of SERT via methylation, protein expression), NPY, and FKBP5 (e.g., snps). Other biomarkers for the autonomic hyperarousal axis may include HPA axis assessment, such as levels of cortisol, and protein biomarkers that may include norepinephrine metabolites. Angiotensin polymorphisms may also be used as biomarkers. Any of these biomarkers may indicate either an autonomic hyperarousal state; some variations may indicate a hypoarousal state.

Examples of biomarkers for the autonomic hyperarousal axis include the serotonin transporter related genes. Serotonin neurotransmitter transporters are the targets of various therapeutic agents used in the treatment of depression and anxiety. The SSRI mechanism of action in depression is mediated by these agents acting as selective antagonists of the serotonin neurotransmitter transporter. Antagonists block uptake and prolong and/or enhance the action of serotonin. SSRI agents, drugs most widely used in depression, selectively block the reuptake of serotonin and result in increased serotonin in the synapse.

a. SLC6A4

The serotonin transporter (5-HTT) is a high affinity carrier protein, localized to the plasma membrane of the presynaptic neuron. The role of 5-HTT is to remove serotonin (5-HT) from the synaptic cleft, resulting in serotonin reuptake into the presynaptic terminus. Elevated synaptic serotonin levels are associated with improved mood; thus the effectiveness of many antidepressant drugs (namely selective serotonin reuptake inhibitors, SSRIs) is thought to be due to their inhibition of the serotonin transporter, thereby reducing serotonin reuptake into the presynaptic terminus, and increasing serotonin availability in the synaptic cleft.

The short (S) allele results in less expression of the active transporter protein compared to the long (L) form. As these genetic differences in the 5-HTT affect both baseline serotonin levels and the availability of the transporter as a target for antidepressant therapy, they can effect the efficacy of antidepressant therapy, the likelihood of side effects, and the nature and extent of depressive symptoms experienced. Studies have shown that compared to L/L patients, those homozygous for the short allele (S/S) are more likely to: (a) respond to antidepressant therapy more slowly, (b) experience adverse drug reactions (ADRs) during antidepressant therapy, and (c) develop major depression following adversity due to a poorer stress response.

In general, L/L individuals report a better and faster response to SSRI therapy than S/S patients. While these L/L individuals may demonstrate appropriate response to SSRI therapy in 2 to 4 weeks, individuals with the short allele (L/S or S/S) may respond to SSRI therapy much more slowly or may benefit from non-selective antidepressants.

In addition to serotonin transporters being targets for anti depressant therapy, it is also recognized that assessment of serotonin transporter activity may be a useful biomarker in psychiatry. Various studies have demonstrated that patients with serotonin transporter short alleles are less likely to respond to SSRI therapy and are also more likely to experience treatment emergent side effects. The specific gene which is tested for, referred to as either the 5HTTLPR or SLC6A4, regulates the rate of serotonin metabolism. This gene controls a receptor located in the synaptic cleft. The receptor binds to serotonin and shuttles it back to the presynaptic neuron, terminating its activity at the post synaptic junction. The binding affinity of this receptor (referred to as SERT) is regulated by hereditary factors related to the length of an allele. Short alleles have reduced binding affinity effects on the serotonin transporter. Conversely, long alleles have better affinity, resulting in a more efficient reuptake process. Thus, the inherited short allele of the serotonin transporter results in more synaptic serotonin and the inherited long allele leads to reduced serotonin in the synapse.

Example of interpretive language in the report for individuals who express short alleles of the serotonin receptor may include:

properties of the short or ‘S’ allele have been associated with decreased transcription of the serotonin transporter and increased vulnerability to major depression and PTSD in the presence of stressors. Subjects homozygous for the s-allele with a significant history of stressful life events exhibit elevated cortisol secretions in response to stress show greater activation in stress-related brain regions such as the hypothalamus and amygdala and may be at higher risk of stress related disorders such as PTSD.

Medical interventions which address heightened physiological arousal in association with serotonin transporter variants should be considered. Noradrenergic agents which demonstrate both an anti anxiety and anti depressive effect may be considered for these individuals. Potential agents which can be regarded may include Mirtazepine, Venflaxaine and Tianeptine.”

b. FKBP5

FKBP5 regulates the cortisol-binding affinity and nuclear translocation of the glucocorticoid receptor. FKBP5 is a glucocorticoid receptor-regulating co-chaperone of hsp-90 and plays a role in the regulation of the hypothalamic-pituitary-adrenocortical system and the pathophysiology of depression.

FK506 regulates glucocorticoid receptor (GR) sensitivity. When it is bound to the FKBP5 receptor complex, cortisol binds with lower affinity and nuclear translocation of the receptor is less efficient. FKBP5 expression is induced by glucocorticoid receptor activation, which provides an ultra-short feedback loop for GR-sensitivity.

Changes in the hypothalamic-pituitary-adrenocortical (HPA) system are characteristic of depression. Because the effects of glucocorticoids are mediated by the glucocorticoid receptor (GR), and GR function is impaired in major depression, due to reduced GR-mediated negative feedback on the HPA axis. Antidepressants have direct effects on the GR, leading to enhanced GR function and increased GR expression.

Polymorphisms the gene encoding this co-chaperone have been shown to associate with differential up-regulation of FKBP5 following GR activation and differences in GR sensitivity and stress hormone system regulation. Alleles associated with enhanced expression of FKBP5 following GR activation, lead to an increased GR resistance and decreased efficiency of the negative feedback of the stress hormone axis. This results in a prolongation of stress hormone system activation following exposure to stress. This dysregulated stress response might be a risk factor for stress-related psychiatric disorders.

Various studies have identified single nucleotide polymorphisms (SNPs) in the FKBP5 gene associated with response to antidepressants, and one study found an association with diagnosis of depression. Polymorphisms at the FKBP5 locus have also been associated with increased recurrence risk of depressive episodes.

In fact, the same alleles are over-represented in individuals with major depression, bipolar disorder and post-traumatic stress disorder.

Individuals homozygous for the TT-genotype at one of the markers (rs1360780) reported more depressive episodes and responded better to antidepressant treatment.

c. 5HT1A

Quantitative genetic studies have found considerable variability in the activity of the hypothalamus-pituitary-adrenal (HPA) axis in response to stress. The HPA axis is regulated by a neuronal network including the amygdala, which is influenced by the effects of the −1019 G/C polymorphism in the 5HT1A (HTR1A) gene. Reductions in postsynaptic 5-HT1A receptor binding in amygdala is correlated with untreated panic disorder. Several single nucleotide polymorphisms have been described for 5-HT1A receptor gene. The HT1A C(−1019)G polymorphism is located in a transcriptional regulatory region and G allele and/or G/G of 5-HT1A C(−1019)G polymorphism genotype was found to be associated with major depression, anxiety and suicide risk.

d. NPY

Anxiety is integrated in the amygdaloid nuclei and involves the interplay of the amygdala and various other areas of the brain. Neuropeptides play a critical role in regulating this process. Neuropeptide Y (NPY), a 36 aa peptide, is highly expressed in the amygdala. It exerts potent anxiolytic effects through cognate postsynaptic Y1 receptors, but augments anxiety through presynaptic Y2 receptors.

The activity of NPY is likely mediated by the presynaptic inhibition of GABA and/or NPY release from interneurons and/or efferent projection neurons of the basolateral and central amygdala. A less active NPY rs16147-399C allele conferred slow response after 2 weeks and failure to achieve remission after four weeks of treatment. The rs16147 C allele was further associated with stronger bilateral amygdala activation in response to threatening faces in an allele-dose fashion.

### Executive Brain, Emotional Valence and Reward System

The executive brain function and the emotional valence and reward systems are combined into a single axis, though in some variations they may be separated out into two axes. This axis is generally concerned with regulation of cortical/frontal lobe and limbic and subcortical (e.g., ventral striatal and nucleus acumbens) dopamine systems. Biomarkers may include: COMT (e.g., snps); sigma receptor (e.g., polymorphisms), Dopamine transporter genes (e.g., SLC6A3), and methylation genes. The methylation genes appear to be in epistasis with COMT in such a fashion that low methylation states may reduce dopamine by disinhibiting COMT degradation of catecholamines. These biomarkers may be thought of as markers for working memory, executive brain function and attentional processes.

DNA methylation is associated with gene silencing, stress, and memory. The catechol-O-methyltransferase (COMT) Val(158) allele in rs4680 is associated with differential enzyme activity, stress responsivity, and prefrontal activity during working memory (WM). Methylation of the Val(158) allele measured from peripheral tissue is associated negatively with lifetime stress; it interacts with stress to modulate prefrontal activity during WM, such that greater stress and lower methylation are related to reduced cortical efficiency, suggests that stress-related methylation is associated with silencing of the gene, which partially compensates the physiological role of the high-activity Val allele in prefrontal cognition and activity.

Limbic and/or subcortical dopamine regulation is also included in this axis, and biomarkers for this activity may include: DRD2/ANKK1 (e.g., TaqI A polymorphism, which has been suggested to be involved in a reward-related psychiatric disorders). Mesolimbic dopaminergic pathways are modulated by the brain-derived neurotrophic factor (BDNF) and the ankyrin repeat and kinase domain containing 1 (ANKK1) gene (e.g., a biomarker may include the DRD2 Taq Ia/ANKK1 snp) which regulate novelty seeking and harm avoidance through dopaminergic mesolimbic pathways. These pathways are is associated with a relatively low D(2) receptor density in the striatum. Additional biomarkers implicated in regulation of limbic and/or subcortical dopamine may include: DRD2 genes and the like.

a. COMT

COMT is an enzyme involved in the degradation of dopamine, predominantly in the frontal cortex. Several polymorphisms in the COMT gene have been associated with poor cognition, diminished working memory, and increased anxiety as a consequence of altered dopamine catabolism. Suitable COMT gene polymorphisms include, e.g., a polymorphism in a Catechol O-methyltransferase (COMT) gene, the major enzyme determining prefrontal dopamine levels, which has a common functional polymorphism (val(158)met) that affects prefrontal function and working memory capacity and has also been associated with anxiety and emotional dysregulation. A single nucleotide polymorphism in the COMT (Val158/108Met) gene affects the concentration of dopamine in the prefrontal cortex.

The COMT 158val/val genotype confers a significant risk of worse response after 4-6 weeks of antidepressant treatment in patients with major depression. There is a negative influence of the higher activity COMT 158val/val genotype on antidepressant treatment response during the first 6 weeks of pharmacological treatment in major depression, possibly conferred by decreased dopamine availability. This finding suggests a potentially beneficial effect of an antidepressive add-on therapy with substances increasing dopamine availability individually tailored according to COMT val158met genotype by inhibiting excess COMT activity.

Suggested language of results of COMT variants may include the following:

The functional Val158Met polymorphism in the gene coding for the catechol-O-methyltransferase (COMT), the major enzyme degrading the neurotransmitters dopamine and norepinephrine, has been associated with differential reactivity in limbic and prefrontal brain and may contribute to individual differences in reward-seeking behavior and in predisposition to neuropsychiatric disorders.”

Individual differences in dopamine mediated ventral striatal activity may relate to disruptions in hedonic state and motivation seen in some forms of depression related to lower dopamine signaling in these individuals.”

Medical interventions which address a potential hypodopaminergic state may be considered in individuals with COMT polymorphisms possibly conferred by decreased dopamine availability. This finding suggests a potentially beneficial effect of an antidepressive add-on therapy with substances increasing dopamine availability individually tailored according to COMT val158met genotype.”

Dopamine agonists which can be selectively employed to individuals with this COMT val polymorphism include MAO inhibitors, Buproprion, or a stimulant.”

b. DRD2

Several lines of evidence suggest that antipsychotic drug efficacy is mediated by dopamine type 2 (D(2)) receptor blockade. Six studies reported results for the −141C Ins/Del polymorphism which indicated that the Del allele carrier is significantly associated with poorer antipsychotic drug response relative to the Ins/Ins genotype. These findings suggest that variation in the D(2) receptor gene can, in part, explain variation in the timing of clinical response to antipsychotics and higher risk of weight gain in deletion allele subtypes of the DRD2 gene.

Suggested language in an interpretive report may include the following:

Abnormalities in the binding potential of the dopamine D(2) receptor have been associated with psychiatric disorders including schizophrenia, autism and OCD. DRD2 receptors are expressed in the orbital cortex and caudate nucleus, regions of the brain associated with perseverative cognitive and emotional responses in depression studies have demonstrated lower density of dopamine D2 receptor (DRD2) in subjects without Del alleles of the −141C Ins/Del polymorphism in DRD2 gene promoter region than in those with one or two Del alleles. Patients without Del allele demonstrate a higher percentage of improvement in anxiety-depression symptoms than those with Del allele after treatment with dopamine antagonists, but prospective clinical trials are required to firmly establish this relationship.

Medical interventions which address imbalances of dopamine binding to the DRD2 receptor in the caudate and orbital cortex should be considered in individuals with DRD2 variants.”

c. SLC6A3

A genetic polymorphism identified as a potential risk factor for hypodopaminergic conditions such as ADHD and substance abuse is a 40-bp variable number of tandem repeats (VNTR) polymorphism within the 3′ untranslated region (UTR) of SLC6A3 gene. It has two common alleles designated as nine-repeat (9-repeat) and ten-repeat (10-repeat), which have been suggested to influence SLC6A3 expression and, thereby, dopamine regulation.

SLC6A3 has been assumed to have a crucial role in regulating the cortical signal-to-noise ratio via its influence on prefrontal pyramidal neurons through regulation of DA volume transmission on the surrounding GABA-inhibitory neurons. It may also influence the cortical signal-to-noise ratio indirectly through effects in the striatum, which regulates activity within the cortico-striato-thalamo-cortical pathway.

Two SPECT studies showed that carriers of at least one nine-repeat allele exhibited increased striatal DAT availability and a striatal hypodopaminergic state due to heightened striatal DAT availability. Association have been made between the DAT1 9R allele with adult ADHD, and significant associations were observed between SLC6A3 VNTR A9 and alcoholics

### Memory and Cognition.

Disruptions in the memory and cognition axis may indicate proneness to paroxysmal disturbances, irritability, instability, neurodegenerative vulnerability and the like. The memory and cognition axis may be probed with biomarkers to the glutamatergic pathway (e.g., NMDA and AMPA receptors) as well as calcium and sodium ion channels. For example, Cav1.2 is thought to be important in modifying the effects of: synaptic activity on cell survival, synaptic plasticity, MAPK pathway activation and critical pathways involved in learning and memory. Intracellular calcium levels are regulated specifically by Cav1.2 which play a role in learning and memory via mediating the downstream effects of glutamate neurotransmission. Cav1.2 mRNA levels increase following repeated amphetamine administration, and CACNA1C are elevated in postmortem brains from BP patients.

Preclinical and clinical studies support a role for CACNA1C in mood disorder pathophysiology, with potentially a sex specific effect in females. Biomarkers implicated in regulation of glutamate may include calcium channel snps (e.g., rs 2370419, 1006736). The presence of these biomarkers may suggest that treatment with a calcium channel antagonist may be therapeutic in such patients. Other biomarkers may include sodium channel snps, whose presence may suggest treatment with sodium channel antagonist may be indicated. Other genes that may be tested as biomarkers include: ANK3 (rs 10994336), SCN1A, and BDNF (val66met).

Examples of biomarkers and interpretive text in the memory and cognition axis include:

a. CACNA1C

The calcium ion is one of the most versatile, ancient, and universal of biological signaling molecules, known to regulate physiological systems at every level from membrane potential and ion transporters to kinases and transcription factors. Disruptions of intracellular calcium homeostasis underlie a host of emerging diseases, the calciumopathies. Cytosolic calcium signals originate either as extracellular calcium enters through plasma membrane ion channels or from the release of an intracellular store in the endoplasmic reticulum (ER) via inositol triphosphate receptor and ryanodine receptor channels. Therefore, to a large extent, calciumopathies represent a subset of the channelopathies, but include regulatory pathways and the mitochondria, the major intracellular calcium repository that dynamically participates with the ER stores in calcium signaling, thereby integrating cellular energy metabolism into these pathways, a process of emerging importance in the analysis of the neurodegenerative and neuropsychiatric diseases.

Molecular genetic analysis offers opportunities to advance our understanding of the nosological relationship between psychiatric diagnostic categories in general, and the mood and psychotic disorders in particular. The CACNA1C gene encodes one subunit of a calcium channel. Results suggest that ion channelopathies may be involved in the pathogenesis of bipolar disorder, schizophrenia and autism with an overlap in their pathogenesis based upon disturbances in brain calcium channels.

CACNA1C encodes for the voltage-dependent calcium channel L-type, alpha 1c subunit. Gene variants in CACNA1 are associated with altered calcium gating and excessive neuronal depolarization. CACNA1 polymorphisms have been associated with increased risk of bipolar disease

Psychiatric disease phenotypes, such as schizophrenia, bipolar disease, recurrent depression and autism, produce a constitutionally hyperexcitable neuronal state that is susceptible to periodic decompensations. The gene families and genetic lesions underlying these disorders may converge on CACNA1C, which encodes the voltage gated calcium channel.

These findings suggest some degree of overlap in the biological underpinnings of susceptibility to mental illness across the clinical spectrum of mood and psychotic disorders, and show that at least some loci can have a relatively general effect on susceptibility to diagnostic categories based upon alterations in calcium signaling.

Medical interventions which address heightened neuronal depolarization in the hippocampaus in association with calcium channel variants should be considered.

Agents which modulate or exert effects on calcium channels may be preferred agents to use in patients with psychiatric disorders in pts who exhibit these variants, as will be further described in subsequent paragraphs. Such agents may include specific L type voltage gated calcium channel inhibitors such as Nimodipine and the like. They may also include other mood stabilizers, such as Lithium or Valproic acid.

b. ANK3

Another biomarker includes the ANK3 gene (e.g., rs 10994336). Genetic variants in ankyrin 3 (ANK3) have recently been shown to be associated with bipolar disorder (BD). The gene ANK3 encodes ankyrin-G, a large protein whose neural-specific isoforms, localized at the axonal initial segment and nodes of Ranvier, may help maintain ion channels and cell adhesion molecules. ANK3 is essential for both normal clustering of voltage-gated sodium channels at axon initial segments

c. BDNF

Brain-derived neurotrophic factor is a member of the nerve growth factor family. It is induced by cortical neurons and is necessary neurogenesis and neuronal plasticity. BDNF has been shown to mediate the effects of repeated stress exposure and long term antidepressant treatment on neurogenesis and neuronal survival within the hippocampus. The BDNF Val66Met variant is associated with hippocampal dysfunction, anxiety, and depressive traits. Previous genetic work has identified a potential association between a Val66Met polymorphism in the BDNF gene and bipolar disorder. Meta-analysis based on all original published association studies between the Val66Met polymorphism and bipolar disorder up to May 2007 shows modest but statistically significant evidence for the association between the Val66Met polymorphism and bipolar disorder from 14 studies consisting of 4248 cases, 7080 control subjects and 858 nuclear families.

The BDNF gene may play a role in the regulation of stress response and in the biology of depression and the expression of brain-derived neurotrophic factor (BDNF) may be a downstream target of various antidepressants.

Exposure to stress causes dysfunctions in circuits connecting hippocampus and prefrontal cortex. BDNF is down-regulated after stress. Acute treatment with the antidepressants tianeptine reverses stress-induced down-regulation of BDNF. Tianeptine, increases the phosphorylation of Ser831-GluA1. Psychological stress down-regulates a putative BDNF signaling cascade in the frontal cortex in a manner that is reversible by the antidepressant tianeptine. Thus agents which promote BDNF are novel mechanisms to treat stress induced alterations in the limbic system

Activation of AMPA receptors by agonists is thought to lead to a conformational change in the receptor causing rapid opening of the ion channel, which stimulates the phosphorylation of CAMK11/PKC sites and subsequently enhance BDNF expression.

A structural class of AMPA receptor positive modulators derived from aniracetam are called Ampakines Aniracetam and Nefiracetam are neurological agents called ‘racetams’ that are analogs of piracetam. They are regarded as AMPA receptor potentiators and CaMKII agonists.

Small molecules that potentiate AMPA receptor show promise in the treatment of depression, a mechanism which also appears to be mediated by promoting BDNF via CaMKII pathways. Depression is associated with abnormal neuronal plasticity. AMPA receptors mediate transmission and plasticity at excitatory synapses in a manner which is positively regulated by phosphorylation at Ser831-GluR1, a CaMKII/PKC site.

Aniracetam [1-(4-methoxybenzoyl)-2-pyrrolidinone] is a AMPA receptor potentiator that preferentially slows AMPA receptor deactivation. AMPA receptor potentiators (ARPs), including aniracetam, exhibit antidepressant-like activity in preclinical tests. Unlike most currently used antidepressants. Interactions of aniracetam with proteins implicated in AMPA receptor trafficking and with scaffolding proteins appear to account for the enhanced membrane expression of AMPA receptors in the hippocampus after antidepressant treatment. The signal transduction and molecular mechanisms underlying alpha-amino-3-hydroxy-5-methyl-4-isoxazole propionate (AMPA)-mediated neuroprotection evoke an accumulation of brain-derived neurotropic factor (BDNF) and enhance TrkB-tyrosine phosphorylation following the release of BDNF. AMPA also activate the downstream target of the phosphatidylinositol 3-kinase (PI3-K) pathway, Akt. The increase in BDNF gene expression appeared to be the downstream target of the PI3-K-dependent by AMPA agonists and Tianeptine (described below). Thus, AMPA receptors protect neurons through a mechanism involving BDNF release, TrkB receptor activation, and up-regulation of CaMKII which increase BDNF expression.

Olfactory bulbectomized (OBX) mice exhibit depressive-like behaviors. Chronic administration (1 mg/kg/day) of nefiracetam, a prototype cognitive enhancer, significantly improves depressive-like behaviors. Decreased calcium/calmoculin-dependent protein kinase II mediates the impairment of hippocampal long-term potentiation in the olfactory bulbectomized mice. Nefiracetam treatment (1 mg/kg/day) significantly elevated CaMKII in the amygdala, prefrontal cortex and hippocampal CA1 regions. Thus, CaMKII, activation mediated by nefiracetam treatment elicits an anti-depressive and cognition-enhancing.

d. SCN1A

A polymorphism within SCN1A (encoding the α subunit of the type I voltage-gated sodium channel) has been replicated in three independent populations of 1699 individuals. Functional magnetic resonance imaging during working memory task detected SCN1A allele-dependent activation differences in brain regions typically involved in working memory processes. These results suggest an important role for SCN1A in human short-term memory.

Voltage-gated sodium channels have an important role in the generation and propagation of the action potential and consist of an alpha subunit, which forms the ion conduction pore, and two auxiliary beta subunits. The alpha subunit has four homologous domains and different genes (SCN1A through SCN11A) encode different alpha subunits named Nav1.1 through Nav1.9 The SCN1A is expressed in brain regions critical for memory formation, regulates excitability of neuronal membranes and several SCN1A mutations are known to cause a variety of neurological diseases such as familial hemiplegic migraine, Some antiepileptic drugs, such as phenyloin and carbamazepine, bind to voltage-gated sodium channels and genetic variability within SCN1A may predict the response to carbamazepine and phenyloin in patients diagnosed with epilepsy.

Lamotrigine, another antiepileptic drug that binds to voltage-gated sodium channels, is an effective maintenance treatment for bipolar disorder, particularly for prophylaxis of depression, a mental disorder with commonly observed working memory deficits. A recent fMRI study reports that lamotrigine treatment in depressed patients results in increased activation of brain regions typically involved in working memory processes.

Heterozygous individuals of the SCN1a gene (rs 10930201) showed significantly increased brain activations compared with homozygous A allele carriers in the right superior frontal gyrus/sulcus, indicating a potential biomarker for Lamotrogine in these individuals with mood disorder.

e. GRM3

Group II metabotropic glutamate receptors (mGluR2 and mGluR3, also called mGlu2 and mGlu3, encoded by GRM2 and GRM3, respectively) are therapeutic targets for several psychiatric disorders.

Both are G-protein coupled receptors, whose activation inhibits adenylate cyclase and decreases cAMP formation. Their primary functions are thought to be as inhibitory autoreceptors and thence modulation of glutamatergic signaling.

It is also of note that, in human brain, polymorphic variation in mGluR3 impacts on EAAT2 expression(glutamate reuptake) The A/A genotype group exhibits a significant reduction of N-acetylaspartate/creatine levels in the right dorsolateral prefrontal cortex compared to the G carriers. A tendency in the same direction was seen in the left dorsolateral prefrontal cortex and in the white matter adjacent to the prefrontal cortex. GRM3 affects prefrontal function and that variation in GRM3, monitored by SNP rs6465084, affects GRM3 function and may indicated reduced glutamate reuptake by a GLT-1 mechanism.

### Drug Metabolism

Biomarkers implicated in drug metabolism may include: 2D6, 2C19, 3A4, MDR1, and 5HT2C. For example, methylation related genes may be biomarkers in this axis.

a. MTHFR

The 5,10-methylenetetrahydrofolate reductase (MTHFR) is a key enzyme for intracellular folate homeostasis and metabolism. Methylfolic acid, synthesized from folate by the enzyme MTHFR, is required for multiple biochemical effects in the brain. A primary role involves the synthesis of dopamine in the brain. Folic acid deficiency results in fatigue, reduced energy and depression. Low folate blood levels are correlated with depression and polymorphisms of the MTHFR gene are closely associated with risk of depression.

MTHFR irreversibly reduces 5-Methyltetrahydrofolate which is used to convert homocysteine to methionine by the enzyme methione synthetase. The c677T SNP of MTHFR has been associated with increased vulnerability to several conditions and symptoms including depression.

Nucleotide 677 in the MTHFR gene has two possibilities: C or T. 677C (leading to an alanine at amino acid 222); 677T (leading to a valine substitution at amino acid 222) encodes a thermolabile enzyme with reduced activity. The degree of enzyme thermolability (assessed as residual activity after heat inactivation) is much greater in 677TT individuals (18-22%) compared with 677CT (56%) and 677CC (66-67%).

Suitable MTHF gene polymorphisms include polymorphisms in the 5,10-methylenetetrahydrofolate reductase (MTHFR) gene, including MTHFR C677T and its association with common psychiatric symptoms including fatigue and depressed mood. These symptoms are proposed to be due to hypomethylation of enzymes which breakdown dopamine through the COMT pathway. In this model, COMT is disinhibited due to low methylation status, resulting in increased dopamine breakdown.

For unipolar depression, the MTHFR C677T polymorphism has been well described and validated.

A recommendation to prescribe various folic acid modulating agents, particularly in individuals with MTHFR polymorphisms, may be part of the interpretive language of our report. Other markers of metabolism, including an analysis of gene polymorphisms associated with hepatic metabolism of psychotropic drugs, have previously been disclosed.

### Example 1

### Treatment Resistant Depression (TRD)

In some variations, the articles of manufacture described herein are generally reports including information of patient-specific biomarkers that is relevant to treatment of treatment resistant depression. The inventor has identified a small sub-set of biomarkers that may be important for understanding in order to best treat TRD. This sub-set of biomarkers may be selected from the four axes discussed above (including the three pharmacodynamic axes and the pharmacokinetic axis), and may represent a subset of biomarkers representing these axes. A patient's genotype for all or a major subset of these six members of this TRD epistatic group (e.g., five of the six, four of the six, three of the six) may provide sufficient information to a medical practitioner to accurately guide treatment. Although information about other genetic loci may be helpful, these six members may be of enhanced importance because they (alone and in combination) offer insight into the patient's specific drug response to TRD. By identifying the specific subset of genes related to the neurochemical imbalance involved in TRD, the treatment choice may be intelligently applied based on the genotype of these genes.

In the reports and methods described herein, the six core members of the TRD include the six genetic loci (forming a core epistatic group) from across the different axes described above: rs25531 variant of 5-HTTLPR (functional variant of a single-nucleotide polymorphism of Serotonin Transporter); MTHFr (variants in methylenetetrahydrofolate reductase); COMT (variants in Catechol Methyl Transferase); DRD2 (variants of Dopamine receptor D2); CACNA1C (polymorphism of L type voltage gated calcium channel); 2D6 (polymorphism of cytochrome p450). In this example, members of this core group are relevant in part because they indicate a possible therapeutic decision. Genes present on the report are those that are relevant to patient treatment outcome, including the avoidance of side effects, increasing effectiveness of drug therapies, or the like. Drugs such as psychotropic agents are of particular interest, and the accompanying interpretive comments (if included) maybe related to the influence of one or more of the core epistatic group on such drugs.

As mentioned above, the 12/790,262 application previously incorporated by reference illustrates how single nucleotide polymorphisms (SNPs) in certain genes may be related to subtypes of depression. In this model, certain antidepressants and other psychotropic agents may mediate their effects via inhibition of CaMKII. Such agents may reduce cortical excitability. The decision to employ this class of agents can be assisted by an analysis of gene polymorphisms which are associated with up-regulation of CaMKII.

The multiple functional and neuroanatomical model described above (including the autonomic arousal axis, the emotional valence and reward and executive brain function axis, and the memory and cognition axis and/or the metabolic axis) may be simplified into a excitatory/inhibitory model of CamKII. Neuropsychiatric disorders may be characterized by an imbalance between excitatory and inhibitory systems at the level of neuronal cellular activity. In this manner, focal brain dysfunctionality may be related to either the hypo- or hyper-functionality secondary to excessive inhibitor or excitatory mechanisms. Some neuropsychiatric disorders may arise because of these discrete excitatory/inhibitory imbalances. For example, frontal lobe effects of a COMT val/val polymorphism, discussed above, may result in hyperfunctional activity resulting in reduced working memory. Changes in the excitatory and inhibitory modulation may be viewed through the filter of the CaMKII activity model previously described. Thus, patients with neuropsychiatric disorders may be characterized by an imbalance between excitatory and inhibitory neurotransmission mediated by CaMKII. In one subtype, the target of a therapeutic requires activation, not inhibition, of CaMKII. The identification of these individuals can be determined by an analysis of a second, distinct subset of genes which results in reduced CaMKII activity. Subsequently, psychotropic agents which activate CaMKII are preferentially indicated. Thus, described therein are methods, devices and systems (e.g., assays) for determining if a patient has one or more SNPs effecting the expression of genes that either reduce or increase the activity or expression of genes that ultimately modulate the activity of CaMKII. Further, also described in the 12/790,262 application are methods, devices and systems for providing treatment guidance based on the identification of one or more of these SNPs.

As mentioned, CaMKII is markedly enriched at synapses, where it is involved in the control of synaptic transmission, transmitter release and synaptic plasticity. Alterations of the activity of CaMKII may form the basis of gene, environment and drug related effects on behavioral states.

Communication between cell surface proteins and the nucleus may be integral to many cellular adaptations. In the case of ion channels in excitable cells, the dynamics of signaling to the nucleus are particularly important because the natural stimulus, surface membrane depolarization, is rapidly pulsatile. CaMKII acting near the channel couples local Ca(2+) rises to signal transduction, encodes the frequency of Ca(2+) channel openings, and amplifies molecular signals in the brain.

Calcineurin is a calmodulin (CaM) dependent protein phosphatase recently found to be altered in the brains of patients suffering from schizophrenia and by repeated antipsychotic treatment. Repeated treatment with haloperidol, clozapine or risperidone decrease CaMKIIalpha, whereas increases in this protein were observed in an amphetamine model of the positive symptoms of schizophrenia.

Lithium is widely used in the treatment of bipolar disorder, although its mechanism of action is not fully clear. Lithium down-regulates CaMKIV (enzymatic activity, phospho-Thr 196 and protein expression level) in the hippocampus, indicating the involvement of CaMKIV in the mechanism of action of lithium.

Intracellular calcium influx through NMDA receptors triggers a cascade of deleterious signaling events which lead to neuronal death Inhibitors of Ca(2+)/calmodulin-dependent protein kinase II (CaMKII) prevent the occurrence of apoptosis, suggesting a role for CaMKII in NMDA mediated cell death. These examples are meant to provide instruction on a genetic basis of analysis whether excitatory or inhibitory brain pathways need to be targeted by select psychopharmacological interventions.

This observation thus discloses a new putative site of action and classification of psychotropic drugs, as well as a previously undisclosed explanation on how single nucleotide polymorphisms in various genes are related to subtypes of depression. In this model, certain antidepressants and other psychotropic agents mediate their effects via inhibition of CaMKII. These agents reduce cortical excitability. The decision to employ this class of agents can be assisted by an analysis of gene polymorphisms which are associated with up-regulation of CaMKII.

However, a separate and phenotypically distinct group of patients with neuropsychiatric disorders are characterized by an imbalance in inhibitory neurotransmission. In this subtype, the target of a therapeutic requires activation, not inhibition, of CaMKII. The identification of these individuals can be determined by an analysis of a second, distinct subset of genes which results in reduced CaMKII activity. Subsequently, psychotropic agents which activate CaMKII are preferentially indicated.

Specific interventions based upon these gene clusters are also claimed. These treatments promote inhibitory mechanisms in the CNS based upon their specific effects on the abnormal expression of these genes. These agents may include CaKMIV antagonists.

For example, described in the 12/790,262 application are panel assays to determine the presence of SNPs that up-regulate or inhibit CaMKII activity.

The panel assay may also include an interpretive comment indicating the effect of any identified SNPs on the regulation of CaMKII activity. In some variations, the panel assay includes an interpretive comment suggesting a treatment based on identified SNPs.

In general, and SNP indicator indicates the presence or absence of an SNP from a tissue sample. The SNP indicator may be based a screening test, such as a genetic screen (e.g., using a PCR-based test) to determine if the SNP is present within the DNA of a particular patient's tissue sample being examined. Any appropriate test for the individual SNP, or a pooled test for multiple SNPs may be used as part of the methods, kits, assays and systems. As mentioned, the SNP indicators may comprise one or more PCR-based assays. An SNP indicator may be included a report (e.g., visual, oral, printed, electronic, or the like), and may indicate the presence or absence of the particular SNP. The SNP indicator may indicate if the SNP is homozygous or heterozygous.

For example, in some variations, the SNP indicator indicates an SNP that alters the function or expression of genes involved in the stress response, particularly the threshold of activation of the amygdala and hypothalamus. In some variations, the SNP indicator indicates an SNP that alters the function or expression of genes related to autonomic activation pathways. In some variations, the SNP indicator indicates an SNP that alters the function or expression of the glutamate metabolism pathway(s), which relates to cognition and long term potentiation. In some variations, the SNP indicator indicates an SNP that alters the function or expression of genes associated with executive brain function which includes attentional and motivational behavioral states.

### Example 2

### TRD report

The reports described herein may simplify the potentially complex and confusing application of personalized medicine for the treatment of depression (and particularly TRD) by providing a simplified and concise personalized diagnostic report that selects and organizes the relevant genotypic and phenotypic information in a manner that emphasizes only those aspects which are relevant to the treatment of depression (e.g., TRD); the report may also emphasize relevant (core) epistatic members, while omitting or separating out non-core genotype/phenotype information. The reports may also provide interpretive comments relevant to the drug response based on these core epistatic members. As described in greater detail below, these reports, and particularly the interpretive portion of the reports, may include an indexing or weighting system that provides a confidence level for the provided interpretive comments.

Thus, the dimensional assays described herein are best served by interpretive reports which include not just the results of the biomarker testing, but also give patient-specific guidance in treating the patient. There are multiple ways in which these interpretive reports provide substantial advantages and benefits; the examples included below illustrate the way in which patient care and treatment has benefitted from these tests.

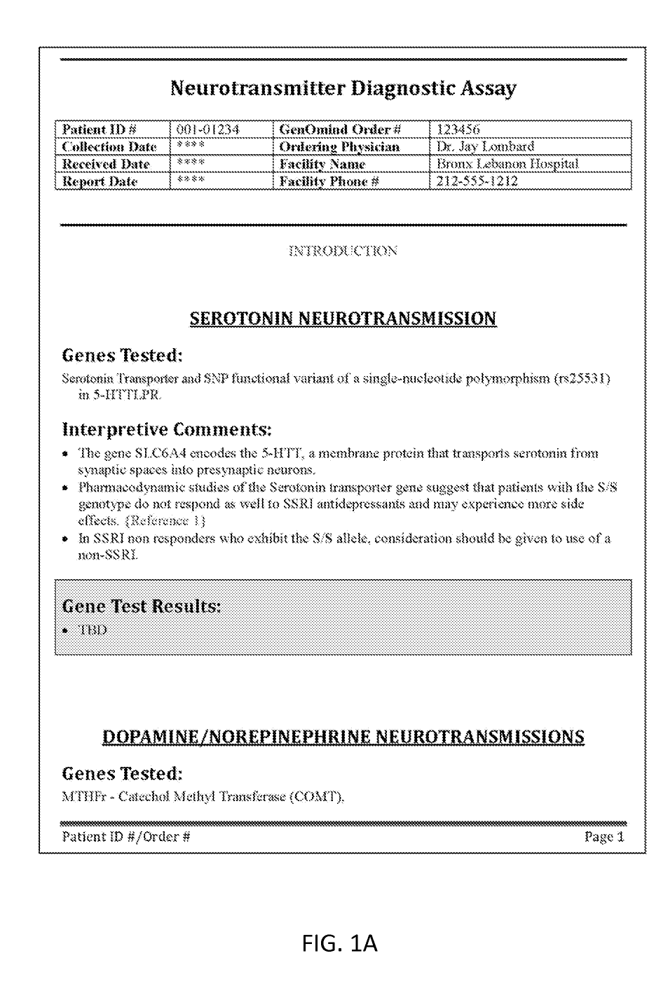

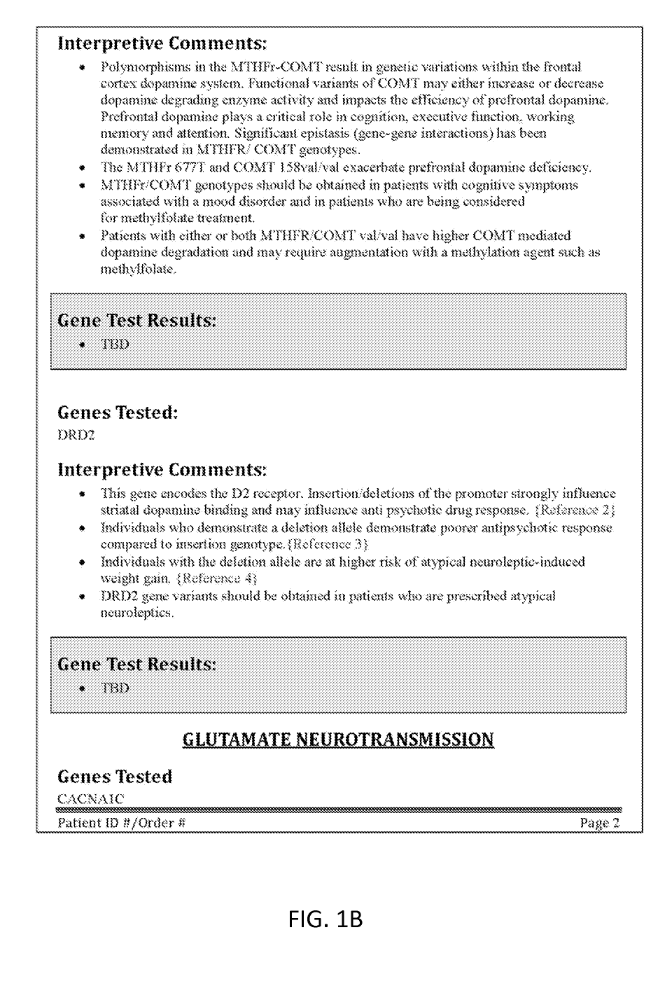

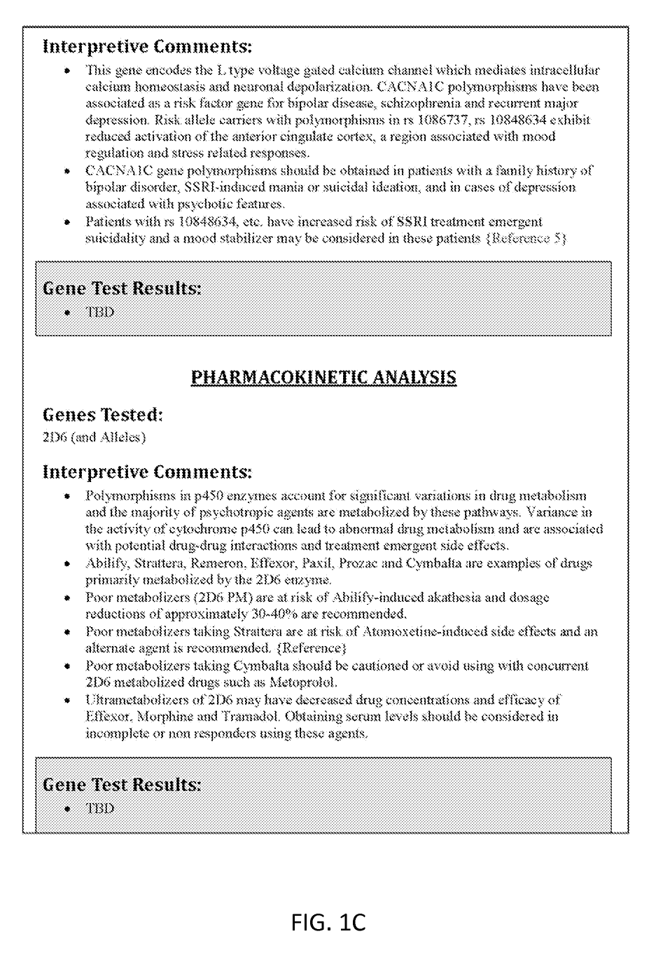

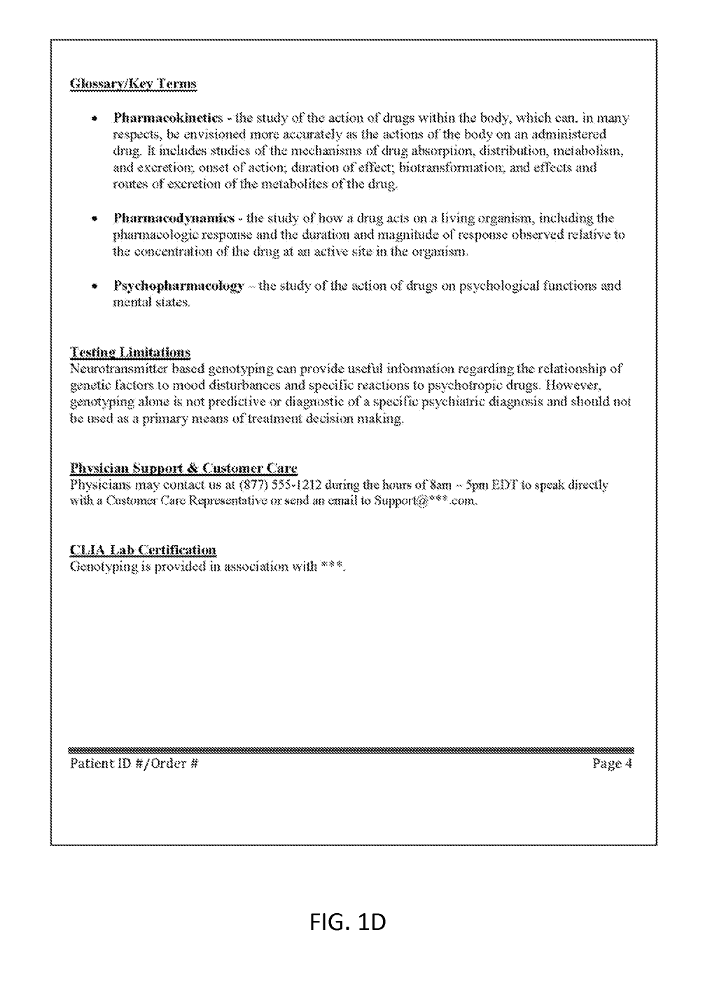

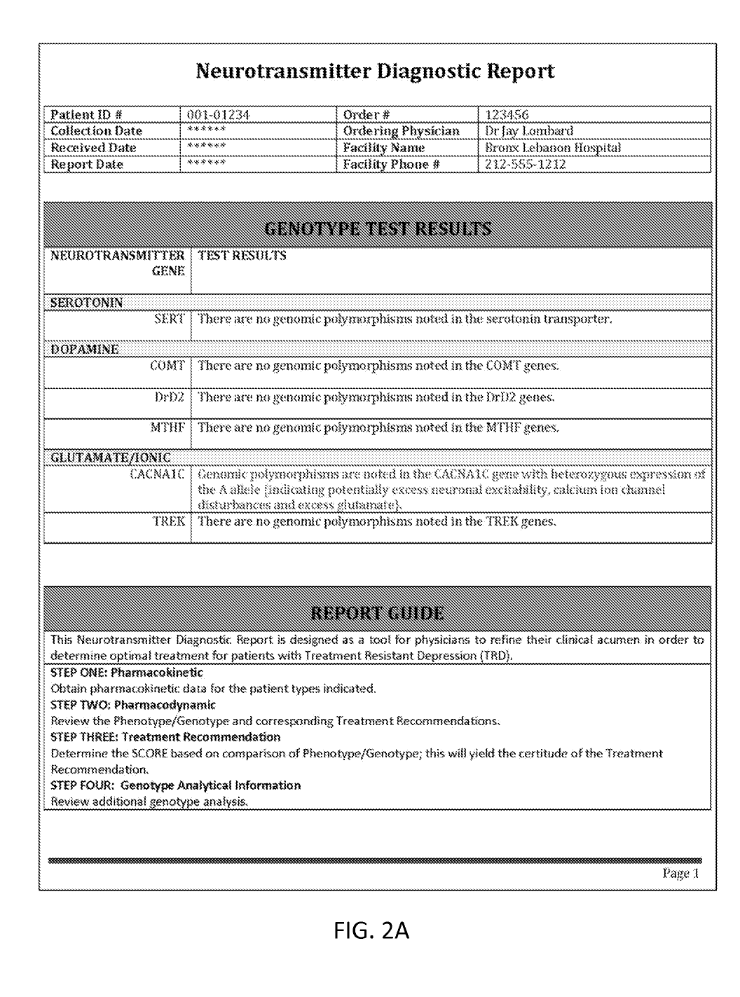

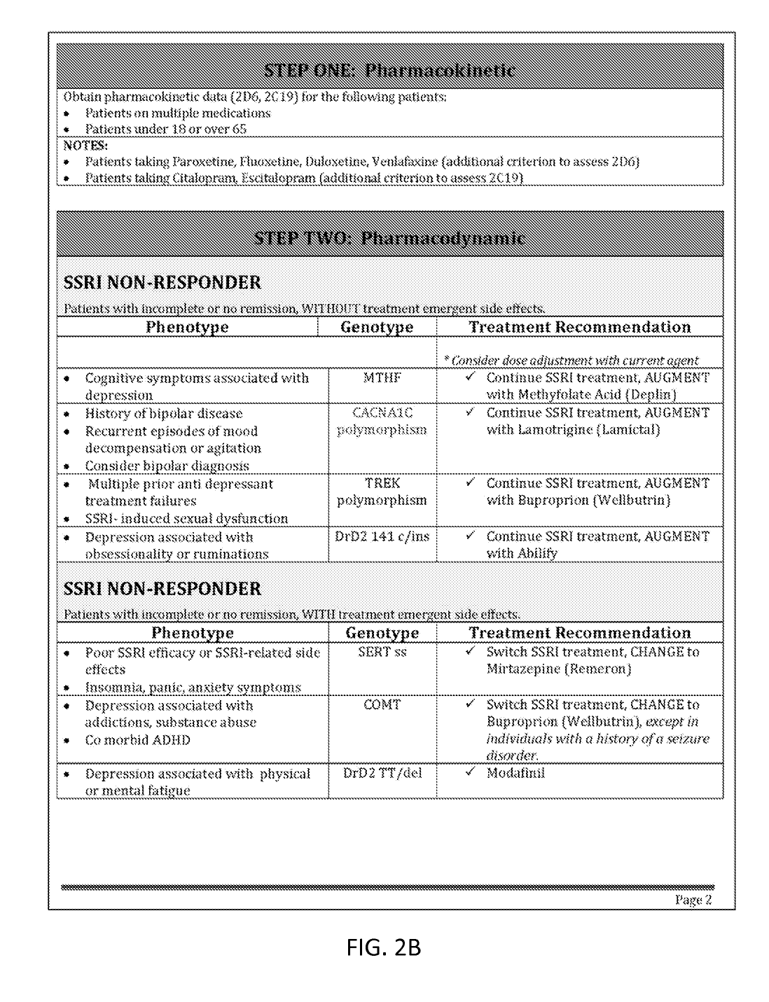

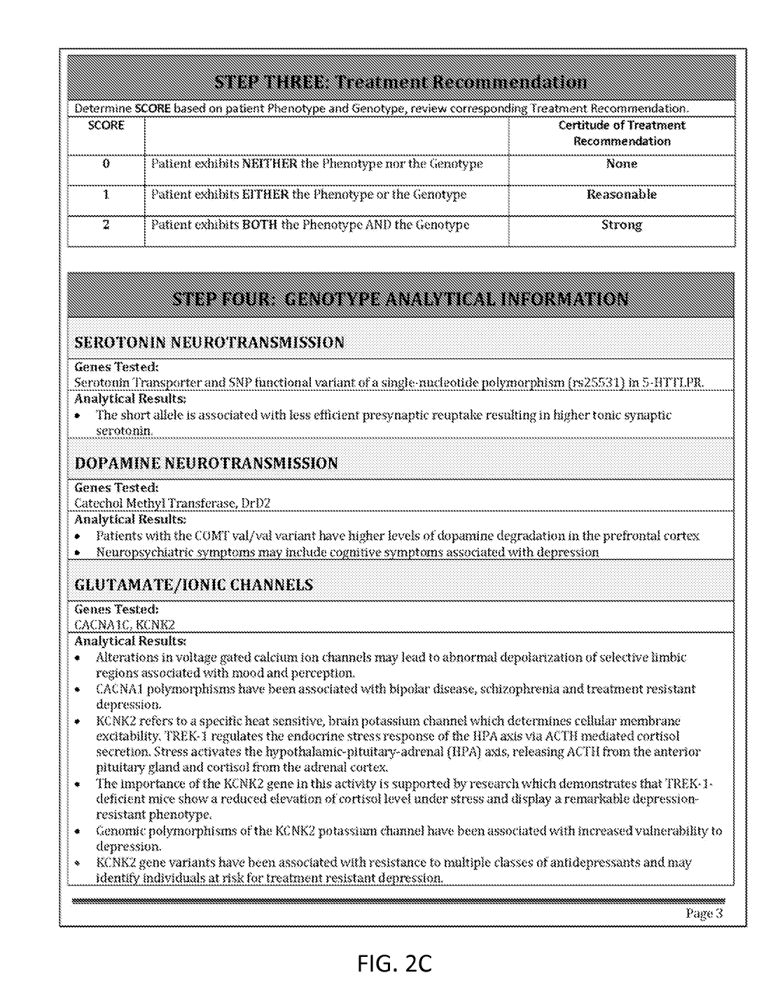

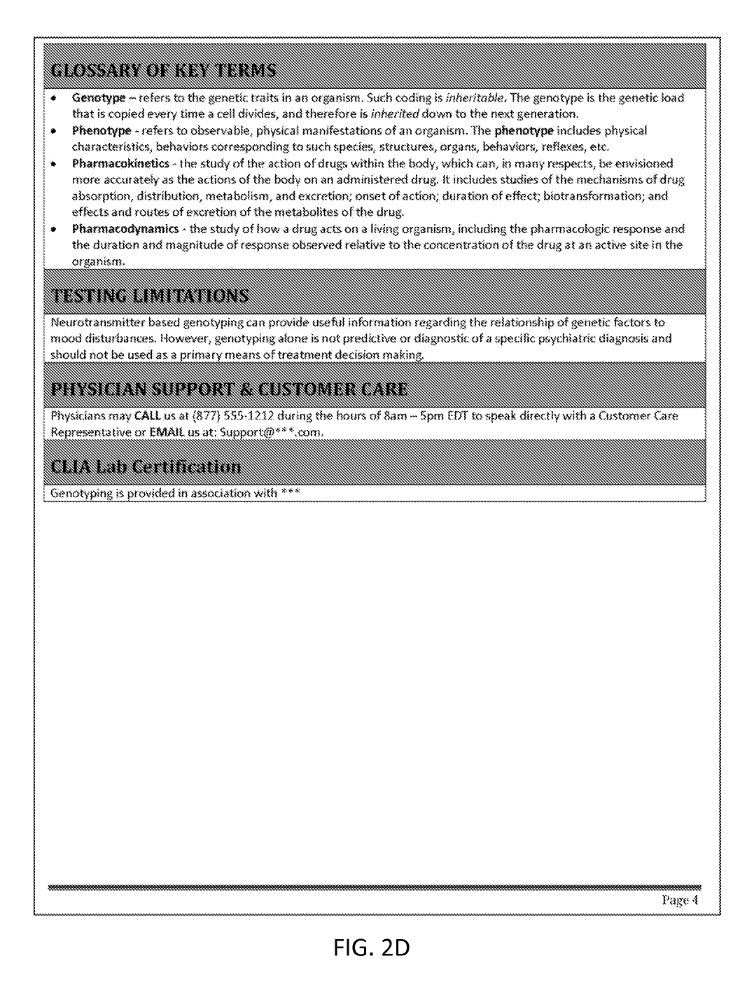

FIGS. 1A-1D and 2A-2D illustrate two examples, respectively, of reports including genetic information that is patient-specific and relevant to treatment of treatment resistant depression. For example in FIGS. 1A-1D, the report is divided up into four sections: serotonin neurotransmission (serotonin transporter and SNP functional variant of a single-nucleotide polymorphism (rs25531) in 5-HTTLPR); dopamine/norepinephrine neurotransmission (MTHFr—Catechol Methyl Transferase (COMT) and DrD2); glutamate neurotransmission (CACNA1C, and the like such as glutamate transporter genes); and pharmacokinetic analysis (2D6, 2c19, 3a4 and the like). In these examples, generic interpretive comments describe the function of the genes tested, and the significance of the resulting genotypes. Finally each section indicates the patient-specific genotype result in a box following the interpretive results.

The report shown in FIGS. 1A-1D also includes a glossary of key terms, and may include references, a description of the genetic testing (including testing limitations) and contact information for further descriptions of the testing and/or results.

FIGS. 2A-2D illustrate another variation, in which a slightly different set of genetic loci form the core epistatic group displayed. In this variation the report includes a summary table (FIG. 2A) listing the gene tested (by class in each of three key classes: serotonin, dopamine and gluitamate/ionic) and the patient genotype. This summary is followed by a “report guide” section describing the intent and use of the report. A description of the pharmacokinetic information (e.g., the 2D6 genetic locus) is described in FIG. 2B. This figure also shows the beginning of the section broken down by pharmacodynamic information. In this section the phenotype may be compared with the genotype to provide a suggested treatment. These treatment recommendations may be scored as indicated in FIG. 2C to determine a “certitude” of treatment recommendation. Finally, descriptive information for the various genotypes is provided in FIG. 2C. FIG. 2D includes a glossary of key terms and a description of the testing limitations.

As mentioned above, the reports described herein, in addition to displaying and highlighting the genotype information for all or a subset of the core epistatic group, may include one or more interpretive results.

For example, interpretive results that may be included for the serotonin neurotransmission locus (e.g., the SNP functional variant of a single-nucleotide polymorphism (rs25531) in 5-HTTLPR (serotonin-transporter-linked promoter region of the serotonin transporter gene)) may include descriptions of the gene or region of the gene examined by the genetic test (“the gene SLC6A4 encodes the 5-HTT, a membrane protein that transports serotonin from synaptic spaces into presynaptic neurons”), as well as information specifically relevant to the drug response/treatment response (“pharmacodynamic studies of the serotonin transporter gene suggest that patients with the S/S genotype do not respond as well to SSRI antidepressants and may experience more side effects,” “in SSRI non responders who exhibit the S/S allele, consideration should be given to use of a non-SSRI,” etc.). References may also be provided.

Interpretive comments that may be provided relevant to the dopamine/norepinephrine neurotransmission locus (e.g., the MTHFr—Catechol Methyl Transferase (COMT)) locus may include, for example: a description of the genetic locus and its relevance (“polymorphisms in the MTHFr-COMT result in genetic variations within the frontal cortex dopamine system. Functional variants of COMT may either increase or decrease dopamine degrading enzyme activity and impacts the efficiency of prefrontal dopamine. Prefrontal dopamine plays a critical role in cognition, executive function, working memory and attention. Significant epistasis (gene-gene interactions) has been demonstrated in MTHFR/COMT genotypes.”), as well as information specifically relevant to the drug response/treatment response (“the MTHFr 677T and COMT 158val/val exacerbate prefrontal dopamine deficiency,” “MTHFr/COMT genotypes should be obtained in patients with cognitive symptoms associated with a mood disorder and in patients who are being considered for methylfolate treatment,” “patients with either or both MTHFR/COMT val/val have higher COMT mediated dopamine degradation and may require augmentation with a methylation agent such as methylfolate.”). References may also be provided.

Interpretive comments that may be provided relevant to the DRD2 (the Dopamine receptor D2) of the dopamine/norepinephrine neurotransmission locus may include, for example: a description of the genetic locus and its relevance (“This gene encodes the D2 receptor.”), as well as information specifically relevant to the drug response/treatment response (“Insertion/deletions of the promoter strongly influence striatal dopamine binding and may influence anti psychotic drug response,” “Individuals who demonstrate a deletion allele demonstrate poorer antipsychotic response compared to insertion genotype,” “Individuals with the deletion allele are at higher risk of atypical neuroleptic-induced weight gain,” “DRD2 gene variants should be obtained in patients who are prescribed atypical neuroleptics,”). References may also be provided.

Interpretive comments that may be provided relevant to the Glutamate neurotransmission locus (CACNA1C) may include, for example: a description of the genetic locus and its relevance (“the CACNA1C gene in humans encodes a protein that is a voltage-dependent, L-type, alpha 1C subunit (also known as Cav1.2) of a calcium channel,”), as well as information specifically relevant to the drug response/treatment response (“This gene encodes the L type voltage gated calcium channel which mediates intracellular calcium homeostasis and neuronal depolarization. CACNA1C polymorphisms have been associated as a risk factor gene for bipolar disease, schizophrenia and recurrent major depression. Risk allele carriers with polymorphisms in rs 1086737, rs 10848634 exhibit reduced activation of the anterior cingulate cortex, a region associated with mood regulation and stress related responses,” “CACNA1C gene polymorphisms should be obtained in patients with a family history of bipolar disorder, SSRI-induced mania or suicidal ideation, and in cases of depression associated with psychotic features,” “Patients with rs 10848634, etc. have increased risk of SSRI treatment emergent suicidality and a mood stabilizer may be considered in these patients,”). References may also be provided.

Interpretive comments that may be provided relevant to the specific pharmacokineit locus (2D6) may include, for example: a description of the genetic locus and its relevance (“2DG, or Cytochrome P450 2D6 (CYP2D6), is a member of the cytochrome P450 mixed-function oxidase system, and is involved in the metabolism of xenobiotics”), as well as information specifically relevant to the drug response/treatment response (“Polymorphisms in p450 enzymes account for significant variations in drug metabolism and the majority of psychotropic agents are metabolized by these pathways. Variance in the activity of cytochrome p450 can lead to abnormal drug metabolism and are associated with potential drug-drug interactions and treatment emergent side effects,” “Abilify, Strattera, Remeron, Effexor, Paxil, Prozac and Cymbalta are examples of drugs primarily metabolized by the 2D6 enzyme,” “Poor metabolizers (2D6 PM) are at risk of Abilify-induced akathesia and dosage reductions of approximately 30-40% are recommended,” “Poor metabolizers taking Strattera are at risk of Atomoxetine-induced side effects and an alternate agent is recommended,” “Poor metabolizers taking Cymbalta should be cautioned or avoid using with concurrent 2D6 metabolized drugs such as Metoprolol,” “Ultrametabolizers of 2D6 may have decreased drug concentrations and efficacy of Effexor, Morphine and Tramadol. Obtaining serum levels should be considered in incomplete or non responders using these agents,”). References may also be provided.

In general, the reports described herein highlight key genetic loci forming a previously unrecognized epistatic group that is relevant to the treatment of depression (TRD). By presenting a patient's genotype for the key genetic loci, as well as providing information specific the possible outcomes, the methods and reports described herein may enhance patient care.

The reports described herein may be referred to as articles of manufacture. The reports may be presented as a paper printout, or they may be digital. In digital formats, the reports may include links (e.g., hyperlinks) to references or additional sources. In variations including associated studies or current research findings as part of the interpretive comments and/or indexing/weighting of the information, links or references may be provided.

### Example 3

### Indexing/Weighting

As mentioned above, any of the interpretive reports described herein may include indexing or weighting of the interpretive comments. The various types of interpretive comments that may be included in the report include: physiological significance, association studies, current research findings, pharmacological implications, and the like. The information provided by the interpretive comments may be based on medical and scientific research, including both published and unpublished data.

All or a subset of the interpretive comments may be indexed with an indicator (which may also be referred to as an “index”) providing a confidence level for the interpretive comment. For example, in some variations the interpretive comments may include a description or mention of the results of one or more association studies relevant to the patient's biomarker test results. An index may provide weighting context by indicating the appropriateness of the association study to support the interpretive comment. Thus, the report may indicate after the study mentioned a “grade” applied to the study (or to other interpretive comments) indicating the nature of the study (e.g., multiple studies reporting or supporting the provided association with the biomarker, a meta-analysis of multiple or single genome-wide studies supporting the association, multiple studies supporting the association, and a single study supporting the association). A letter, number, symbol, color, or other grade may be used. In some variations the indexing may rank the confidence level (e.g., having grades A through D, 1-4, etc.), with the strongest support being ranked “highest.”

A key to the indexing or weighting may be provided as part of the report. In some variations.

The indexing or weighting may be directly associated with the interpretive comment in the report. For example, the index may be provided as a subscript, superscript, parenthetical, or other text or visual indicator at the beginning or end of the interpretive comment. The index may also be represented in the display of all or a part of the interpretive comment (e.g., changing the color of the interpretive comment, the font, the size, etc.).

Indexing values for all or a subset of the interpretive comments may be generated manually or otherwise. An indexing value may be assigned based on a formula that weighs the reproducibility of the association, the size of the study supporting the interpretive comment, the type of study supporting the interpretive comment, the publication status of the study (which may include the source, e.g., journal, etc., of the study), a metric of how accepted the association is to those of skill in the art, and the presence of contradictory findings.

### Example 4

### Assay Report

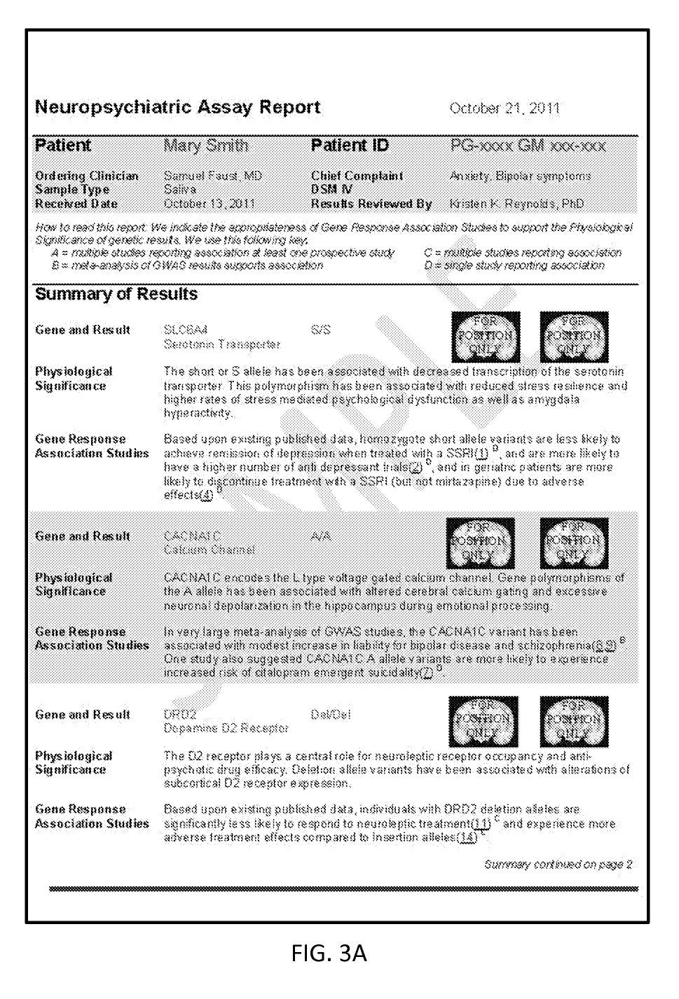

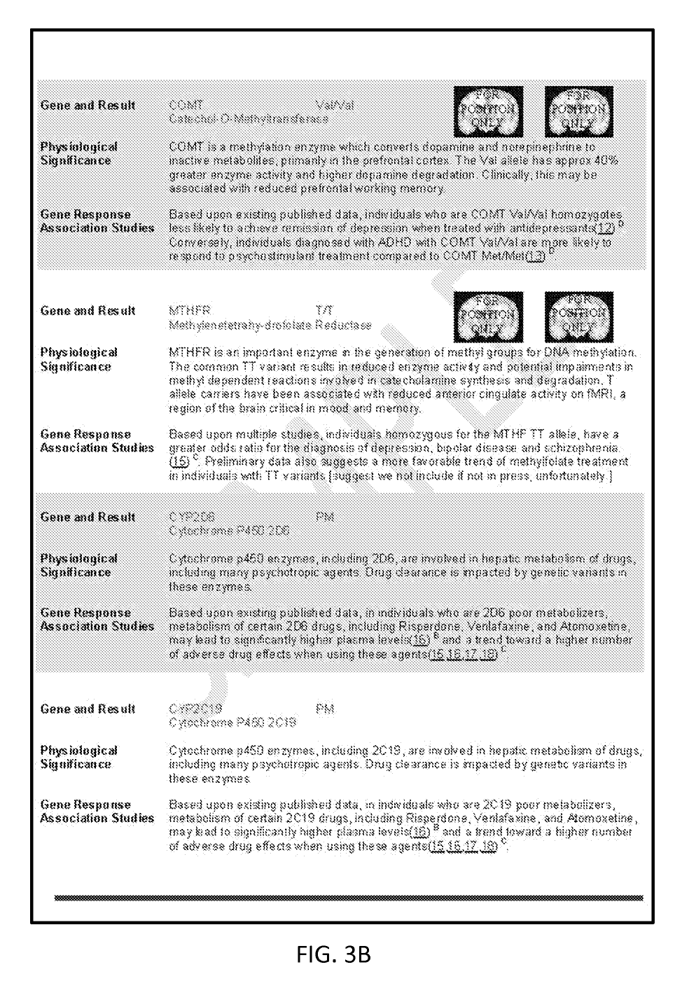

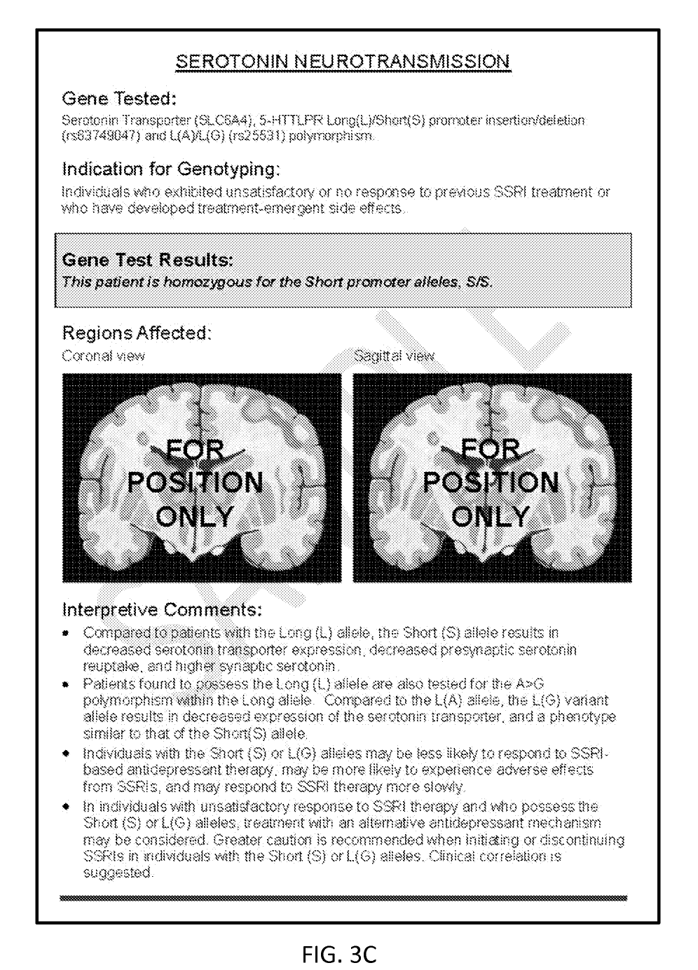

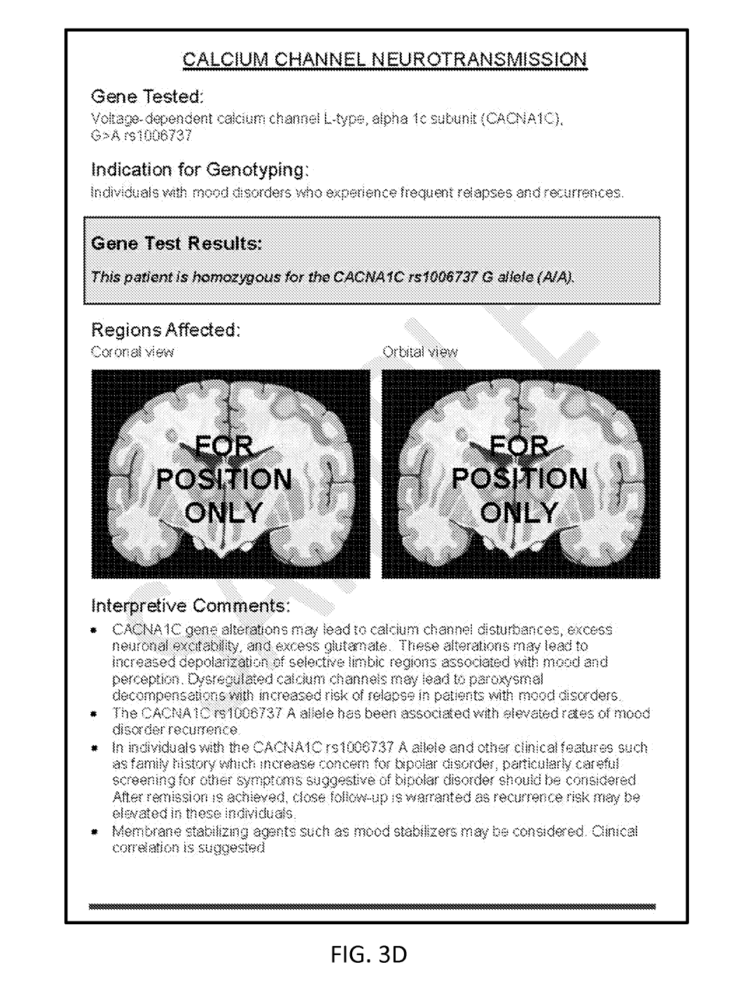

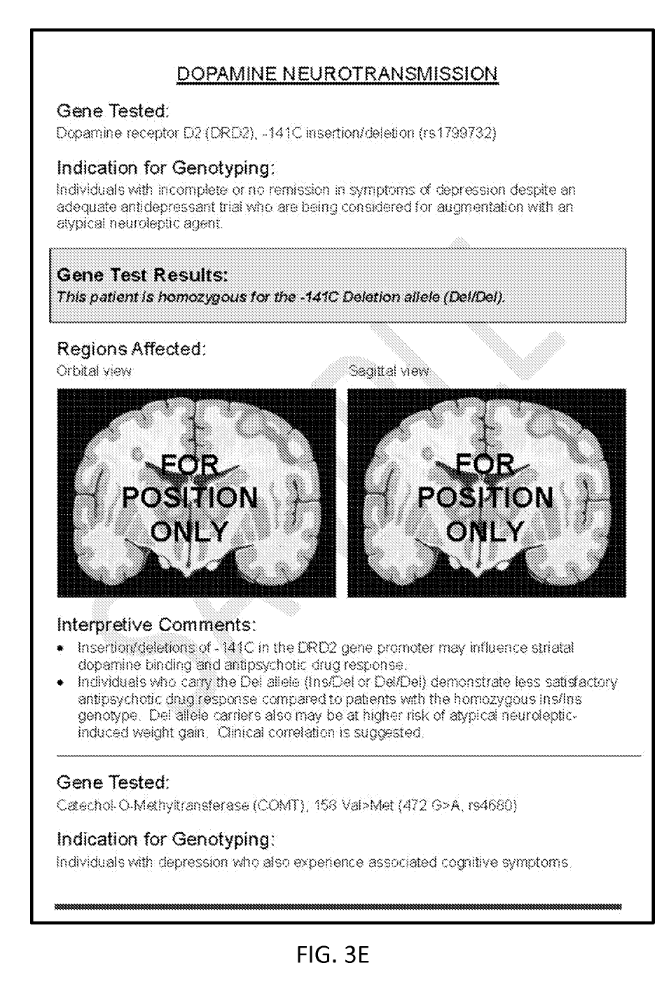

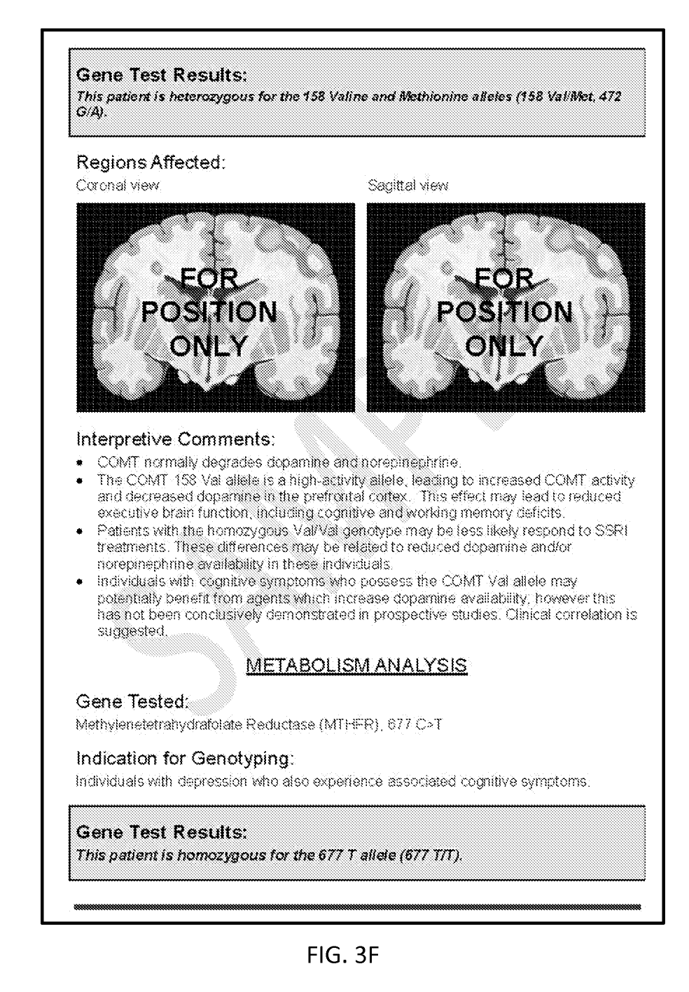

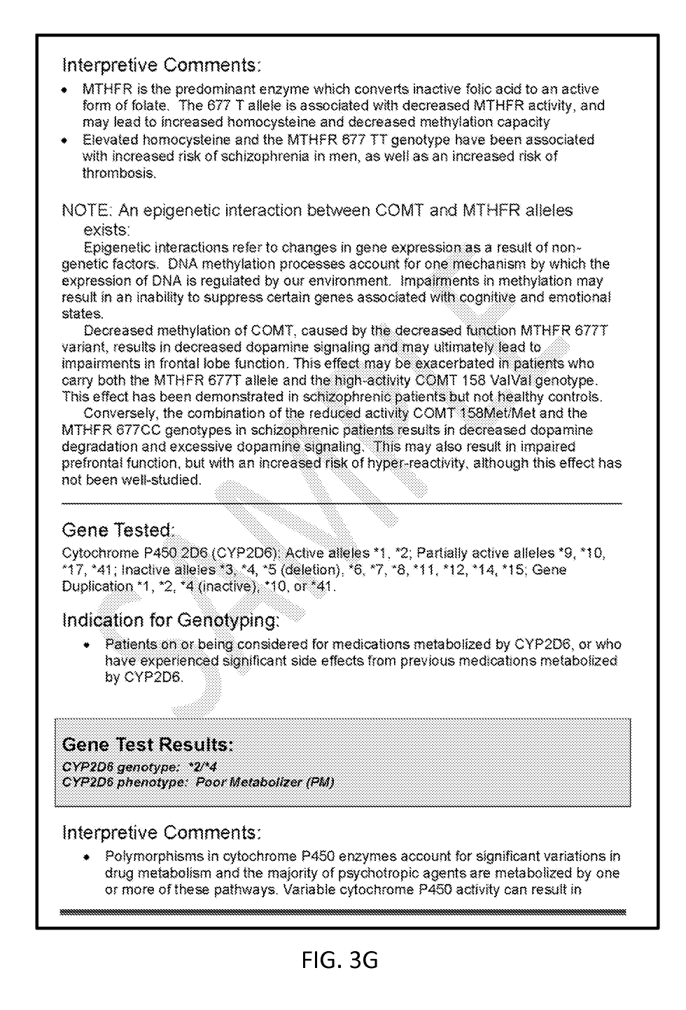

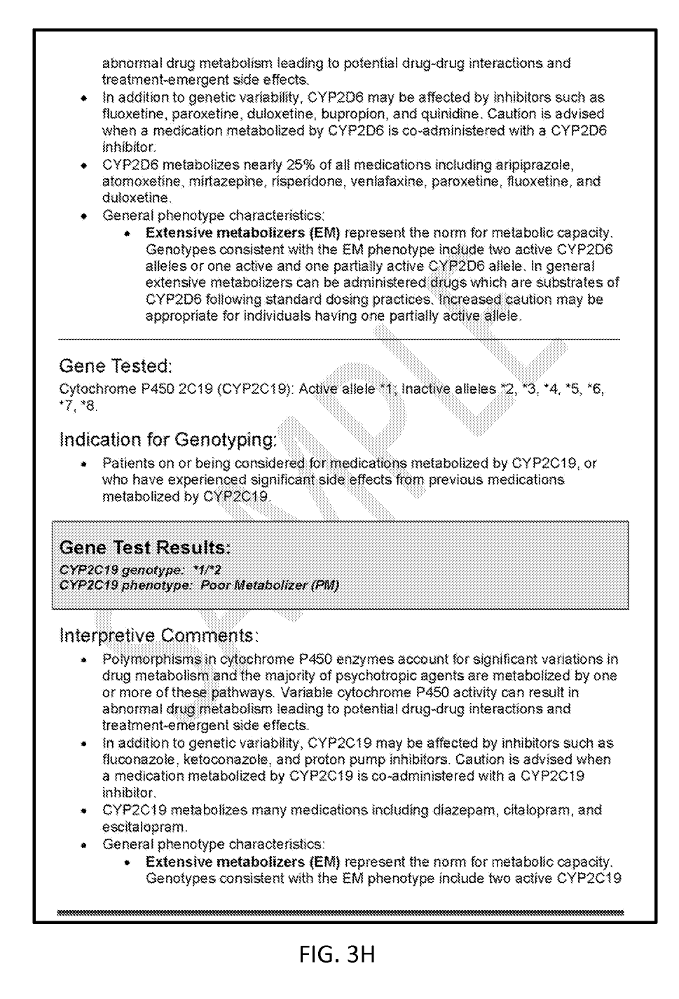

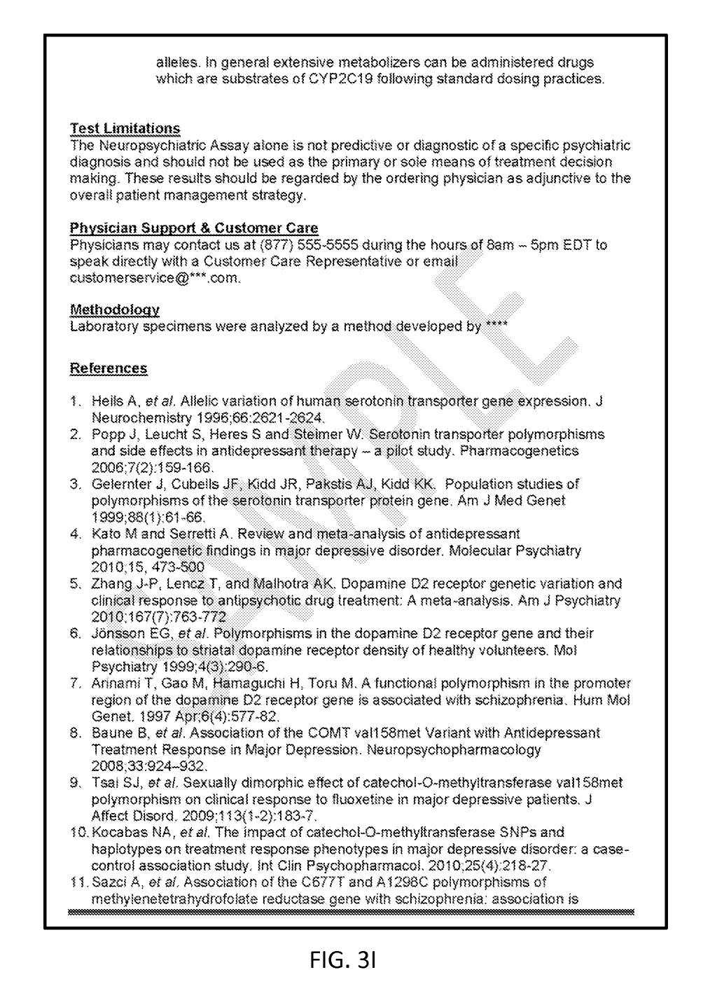

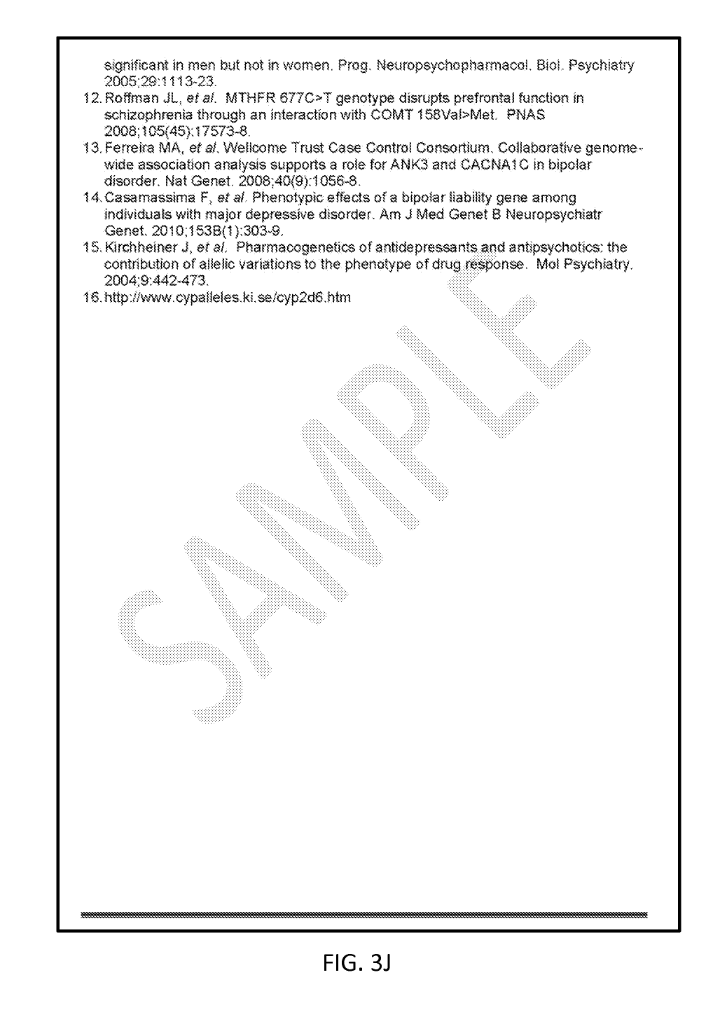

FIGS. 3A-3B illustrate one portion of another variation of an interpretive report. In this example, the report (“Assay Report”) includes a patient identifier (patient name, and/or “patient ID”). The report also indicates the source of the biomarker test results, including the sample type, ordering clinician, receive date, etc. This exemplary assay report also includes information from each of the four axes illustrated above in seven representative biomarkers: SLC6A4, CACNA1C, DRD2, COMT, MTHFR, CYP2D6, and CYP2C19. FIGS. 3C-3J also illustrate additional pages of the interpretive report.

FIG. 3A is the first “page” of the report. In this example the first page includes a brief summary of the patient identifier section, followed by an interpretive key (“How to read this report” section). The interpretive key section describes the index/weighing system used by the report to provide a confidence level to the interpretive comments.

A summary of the results section follows, briefly summarizing the results of each biomarker test as well as the associated interpretive information and confidence index. In this example, the information summarized for each biomarker includes: the biomarker tested (e.g., SLC6A4), a description of what the biomarker is (“Serotonin Transporter”), an indicator of the result (“S/S”), and a brief description of the physiological significance of the test results. An image of the brain region implicated by the biomarker test result may also be shown. In FIGS. 3A and 3B, some of the biomarkers (the psychodynamic biomarkers) include a pair of images (e.g., an exemplary sagittal section and an exemplary coronal section through a brain), illustrating potentially affected brain regions. This spatial mapping may be based on the putative target brain regions indicated earlier.

The example report shown in FIGS. 3A-3B indicates a polymorphism for each examined biomarker. In some variations, when a biomarker result is notable as having a clinically or therapeutically relevant result, the report may highlight the biomarker and test result in some manner. For example, the biomarker test result may be made bigger, may be bolded, may be colored, may be highlighted, may be boxed, etc. In some variations the interpretive results may include an executive summary section that indicates or directs the physician to a potentially relevant result.

In FIG. 3A, the first test result summarized is the SLC6A4 (Serotonin Transporter) gene. The results for this exemplary test report indicate that the hypothetical patient is “S/S” or homozygous for the short allele. The interpretive comments describe the physiological significance and gene response association studies supporting the interpretive comments. In this case, the interpretive comments are also indexed or weighted to indicate a confidence level, and references to supporting documents are provided. For example, the physiological significance of the S/S result for the SLC6A4 biomarker result is described as “the short or S allele has been associated with decreased transcription of the serotonin transporter. This polymorphism has been associated with reduced stress resilience and higher rates of stress mediated psychological dysfunction as well as amygdala hyperactivity.” Further interpretive comments (“gene response association studies”) are also provided, such as “based upon existing published data, homozygote short allele variants are less likely to achieve remission of depression when treated with a SSRI(1) [D], and are more likely to have a higher number of anti-depressant trials(2) [D], and in geriatric patients are more likely to discontinue treatment with a SSRI (but not mirtazapine) due to adverse effects(4) [D].” In this example, the “D” following each of three different statements is an indexing element indicating that only a single study reports the described association (a relatively low confidence rating). The numbers in parenthesis following each statement refer the physician to a reference for further information; the references are listed at the end of the long report (FIGS. 3C-3F).

A similar description for the CACNA1C (calcium channel) gene, DRD2 (dopamine D2 receptor) gene, COMT (Catechol-O-Methyltransferase) gene, MTHFR (Methylenetetrahy-drofolate Reductase) gene, CYP2DG (Cytochrome P4500 2D6) gene, and CYP2C19 (Cytochrome P4500 2C19) gene is also provided.

Following the two-page summary of the results and interpretive comments, additional pages may be provided to go into even greater detail for each biomarker, including additional interpretive comment and enhanced view of potentially affected brain regions.

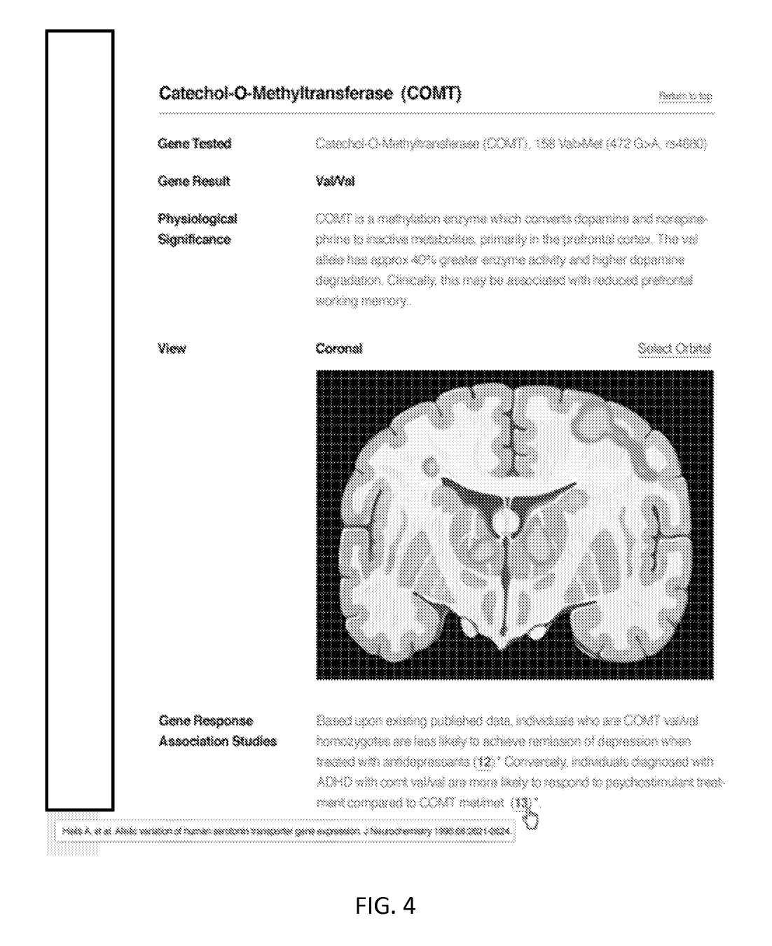

As mentioned above, in some variations, the report may be digitally provided or available. For example, a patient physician may be provided with access to a secure website storing patient information and the results of the assay; software (or firmware, hardware, etc.) running analysis logic may generate an interpretive report such as the one illustrated in FIGS. 3A-3J. FIG. 4 illustrates an alternative format for the report discussed above. FIG. 4 corresponds to the detailed portion of the COMT biomarker described in FIGS. 3E and 3F. FIG. 4 is available as an online (digital) report. In this example, the report is hyperlinked (e.g., to references) and allows toggling between different exemplary views (e.g., coronal and sagittal).

### Example 5

### Biomarkers

Systems and reports for treating (or in some cases, diagnosing) neuropsychiatric disorders may include tests, assays, screens, kits, panels, and the like. In particular, the systems and reports described herein may be used to diagnose or treat depression. In other variations the systems and reports described herein may be used to diagnose or treat other neuropsychiatric disorders. Such systems may examine biomarkers for a specific axis, neurotransmitter pathway, and/or neuroanatomical region. For example, a cluster of biomarkers addressing a particular neurotransmitter pathway (or portion of a pathway) including ion channels, neurotransmitter receptors, etc. may be examined. In addition to such pharmacodynamic biomarkers, pharmacokinetic biomarkers may be included. For example, brain-immune pathways and cerebral metabolism may be probed using one or more biomarkers. Exemplary neurotransmitters, ion channels, or the like are described in Table 1 below, and in other portions of this disclosure. For any of these, the systems described herein may be examined to determine an indicator of the genetic markers (e.g., SNPs), epigenetic markers (e.g., methylation), or protein expression. In addition, also described herein are specific clusters or groups of such genes/encoded proteins that may be examined in combination to provide particular relevance.

TABLE 1
genes for analysis and screening in a neuropsychiatric patient

| | |
|---|---|
| Gene | Exemplary SNP/ALLELE |

Axis I:
genetic markers of neuropsychiatric symptoms associated
with autonomic hyperarousal; symptoms in these individuals,
regardless of diagnosis, may include panic attacks, insomnia,
hypervigilance, fear, increased startle, insomnia

| | |
|---|---|
| SERT (SLC6A4) | Ins/del, rs 25531 |
| 5HT1A (HTR1A) | −1019 C > G, rs 6295 |
| FKBP5 | rs3800373, rs 1360780 |
| NPY | rs 16147 |

Axis II:
genetic markers associated with dopamine dysregulation;
symptoms may include attentional difficulty, poor focus,
reduced ability to plan, impulsivity, motivational issues,
cravings for reinforcing agents

| | |
|---|---|
| COMT | 472 G > A (Val 158 Met), rs 4680 |
| SLC6A3 | VNTR 9/10 repeat |
| DRD2 | −141 C insertion/deletion, rs1799732 |

Axis III:
genetic markers associated with disturbances in excitatory
neurotransmission due to glutamate dysregulation, symptoms
may include heightened irritability, cyclical and recurrent
mood disturbances, paroxysmal complaints

| | |
|---|---|
| CACNA1C | G > A, rs 1006737 |
| ANK3 | rs 10994336 |
| BDNF | G > A (Val 66 Met), rs 6265 |

In some variations of the systems, reports and methods described herein, therapeutic or treatment guidance may be provided based either specifically on the results or score for a particular patient, or more generally presented so that the medical health provider may apply the results of the various screen or panel to a set of guidelines.

For example, Table 3, below provides suggested therapeutic(s) based on the presence of one or more of the genetic, epigenetic or protein assays described herein for the various markers tested.

TABLE 2
exemplary therapeutic/interpretive guidance

| | |
|---|---|
| Axis I, | Norepinephrine reuptake inhibitors |
| generally | (Mirtazepine and the like), Atomexetine, |
| | NRI, SNRIs, Agents which are primarily |
| | anxiolytic, reduce elevated stress related |
| | catecholamines (tianeptine, and the like) |
| Axis II, | Dopamine modulating agents-stimulants, |
| generally | anti-psychotics, buproprione, S |
| | adenosylmethionine, seligiline, and the like |
| Axis III, | Mood stabilizers, such as Lithium, |
| generally | Lamictal, Nimodipine, Vitamin D, |
| | Valproic acid, N acetylcysteine, |
| | magnesium and the like; Racetam based |
| | agents (Aniracetam and the like) |

Additional examples may illustrate the application of the systems, reports and methods described herein for the application of this integrative technology to other neuropsychiatric disorders.

The reports, systems and method described herein may specifically include, discuss, and describe genes having epistatic effects. Epistasis refers to the phenomenon where the effects of one gene are modified by one or several other genes, which are sometimes called modifier genes. The gene whose phenotype is expressed is said to be epistatic. Such relationships may be previously unrecognized, and may aid in the diagnosis and treatment of neuropsychiatric disorders. For example, the inventor has discovered that individuals with COMT val/val in epistasis with MTHF TT may display a phenotype characterized by a subcortical type of mood disorder. These individuals commonly are abulic, dysthymic, and anergic. This phenotype may be expressed secondary to reduced pre frontal dopamine as a consequence of these genes in epistasis, resulting in excess dopamine degradation. Thus, a system, report or method may examine the combination of COMT and MTHF and/or dopamine neurotransmitter pathway genes; one or more of genetic markers, epigenetic markers and/or protein expression may be examined to determine if a patient has or is at risk for the correlated abulic, dysthymic, and anergic phenotype.

In another example, the combination of serotonin short alleles and CACNA1C variants has also been linked by the inventor to a particular phenotype which may be specifically amenable to treatment, either to enhance treatment or to select between available treatments that would otherwise be seemly equivalent based only on the phenotype presented to the physician. For example, SSRI induced mania may be higher in these patients.

We herein further postulate herein that neuropsychiatric subtypes may be based upon imbalances between excitatory and inhibitory mechanisms in the brain. Certain subtypes of depression or dementia are associated with predominant excitatory pathways (such as excess glutamate) which involve abnormal expression of genes and neurotransmitters leading to specific phenomenological behavioral states. In other subtypes of disorders, inhibitory pathways predominate, with abnormal expression of a separate and distinct set of genes and neurotransmitters. Thus, a clinician may be able to ascertain specific subtypes by analyzing both the behavioral and genetic patterns of individuals with neuropsychiatric disorders.

Another example includes looking at mood disorders differentiated by heightened or reduced activity of the amygdala and hypothalamic fight or flight response. In certain individuals, such as those with the SERT short alleles, demonstrate heightened fear response as a result of amygdala excitation. Other genes are likely to evoke similar reductions in the threshold of excitatory pathways, such as FKBP5 and the like. Phenotypically, these patients often demonstrate an imbalance of excess excitation-panic, anxiety, frequent decomensations, and reduced stress resilience. This is in stark comparison to genotypes where there is reduced CNS activity, such as anhedonic states of depression where an activating agent, such as a stimulant, would be more indicated.

In addition to the COMT/MTHFR epistatic relationship, other epistatic sets of genes may also be included, described and discussed in any of the kits, systems, reports and methods.

### Example 6

### Patient Results

The systems, methods and reports described herein have been successfully used during a preliminary testing phase for the neuropsychological assay descried above in Example 4. Neuropsychological patients were tested and the results provided to their treating physician. The physician, using the provided test results and interpretive comments treated the patients as suggested by the interpretive comments. Five example of patients, the results of their neuropsychiatric assay and the resulting physician treatment following receipt of the interpretive assay results are described herein. In general the tests have been surprisingly effective in aiding in patient care, proving (1) biomarker test results that are specific to depression (in these exmpales), and (2) interpretive comments based on the biomarker results that is useful to guide treatment, and (3) indexing/weighting parameters indicating a confidence level.

For example, in a first example, a 16 year old girl was tested (via an in-office saliva collection); the saliva sample was examined as discussed above, and results were provided to generate an interpretive report as described above. In this case, the patient had a history of excessive anxiety starting from the age of three. She was raped at age 14, and shortly after that begin cutting herself, having trouble sleeping. The patient was described as hyper vigilant, and worried about worst case scenarios. She had been prescribed SSRIs, which made her symptoms significantly worse. The results of the neuropsychiatric assay described in here found that her SLC6A4 biomarker indicated that she was homozygous for the short allele (“s/s”) and within normal parameters for the other biomarkers. Based on these results, the clinician elected to prove a noradrenergic agent, as predicted by the interpretive comments. Treatment with the noradrenergic agent resulted in specific and attenuated reduction in her symptoms.

In a second example, a 56 year old man, who has been a very successful businessman, sought the help of a psychiatrist complaining of chronic dysthymia (e.g., low grade persistent depression, also described as “melancholic depression” because of significant vegetative behavior), low libido, and excessive fatigue and low motivation. An assay such as the one described in FIGS. 3A-3J was performed using a patient saliva test. The patient's biomarker results (e.g., genotype) were most remarkable for a COMT val/val polymorphism. Because of these results, the clinician elected to prescribe psychostimulants (targeted as dopamine agonist). The patient reported significant benefits in overall mood, energy and focus following the use of this agent.

In a third example, the patient was a 44 year old woman with a history of migraines and depression, and her depression was cyclical, worsening prior to menstral cycle start. Her treating psychiatrist prescribed an SSRI for depression. The SSRI therapy made her symptoms worse, and was self-discontinued. Following administering of the assay, the test results were passed on to a system/device that generates the customized (patient-specific) interpretive assay report.

The interpretive assay indicated that, though the patient was within normal tolerances for the other biomarker test results, she has a polymorphism in her CACNA1C gene. Based on this previously unsuspected result the treating physician elected to treat her with mood stabilizer (Valporoic acid). This therapy resulted in a dramatic improvement in the patient's migraines and mood.

In a fourth example, a 50 year old female patient sought treatment from a psychiatric complaining of severe depression, precipitated by a divorce request. The patient had a history of depression but had not been medicated for many years. After developing depression she was prescribed a multitude of medications, including SSRI, Abilify, Ativan, etc. Although the drugs did improve her symptoms, she complained that the medications made her feel excessively sedated, and also lead her to gain a significant amount of weight. The dimensional assay was provided as discussed herein, and the results of the biomarker testing used to provide an interpretive report. The most remarkable result of the neuropsychiatric assay was the indication that the patient has an MTHFT variant and normal folic acid levels. Based on this result and the interpretive report, the clinician elected to give high doses of methyl folic acid. Within 2 weeks the patient reported significant improvements in mood and in weight loss. Interesting, this case is of particular interest, because MTHFR variants such as hers have been associated with obesity and methyl folic acid response.

Another example, illustrates the multiaxial nature of the test, showing the assays and reports may successfully be used to interpret polygenic testing via the assays described. For example, a 34 year old girl with bipolar depression, characterized by rapid cycling disorder, and also diagnosed with PTSD, panic attacks and sleep disorder, sought help from her psychiatrist. In the past, she has exhibited an incomplete and unsatisfactory response to prior SSRI trials, including Prozac, Zoloft, and Cymbalta, and while on them experienced mania and agitation. She was subsequently prescribed Abilify (aripiprazole), and some an initial improvement of her symptoms, however, one the drug dosage was raised to 5 mg, the patient developed significant akathesia resulting in discontinuation of the drug. The neuopsychiatric assay was performed, and her physician was informed of the results, which indicated primarily that she was heterozygous for the short transporter (SLC6A4) allele, and also has a DRD2 deletion allele, and a calcium channel variant (CACN1C). Based on these results, she was prescribed lithium, and within a short amount of time experiences a profound improvement.

As discussed above, the prescription of mood stabilizers would be expected to have adverse side effects to antipsychotics because of a deletion allele, and was an incomplete responder to SSRIs because of the DRD2 deletion. Further, the presence of a calcium channel snp predicts that the patient would be at a heighted vulnerability to bipolar disorders. On this basis alone, the decision to prescribe a mood stabilizer is in keeping with the interpretive comments.

In all of the case histories described briefly above, the interpretive results suggested a course of action which was strongly and surprisingly effective in treating neuropsychiatric disorders.

While the reports, methods of forming them, systems, and methods for using them, have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A method of presenting patient-specific pharmacodynamics information relevant to the treatment of a neuropsychiatric disorder, the method comprising:
providing a patient identifier;
presenting a description of a biomarker test result specific to the patient;
presenting an interpretive analysis of the neurophysiological significance of the biomarker test result for the patient, wherein the interpretive analysis comprises pharmacodynamics information; and
presenting a weighted index of confidence level for the interpretive analysis.

2. The method of claim 1, wherein the neuropsychiatric disorder is depression.

3. The method of claim 1, wherein the neuropsychiatric disorders is selected from the group including: treatment resistant depression, bipolar depression, anxiety disorders, dementia, autism, and ADHD.

4. The method of claim 1, further comprising presenting descriptions of biomarker test results specific to the patient for a plurality of biomarkers.

5. The method of claim 4, further comprising presenting an interpretive analysis of the neurophysiological significance of each of the biomarker test results for the patient.

6. The method of claim 1, wherein the interpretive analysis further comprises the physiological significance of the biomarker test result for the patient.

7. The method of claim 1, wherein the interpretive analysis further comprises a description of published studies describing similar biomarker test results.

8. The method of claim 1, wherein presenting a weighted index of confidence level comprises alphanumerically indexing all or a portion of the interpretive analysis with a score indicating the type and/or number of studies supporting the interpretive analysis.

9. The method of claim 1, wherein presenting an interpretive analysis comprises indicating possible drug responses.

10. The method of claim 1, further comprising presenting a description of a biomarker test results for a pharmacokinetic biomarker.

11. The method of claim 1, further comprising presenting a visual representation of a brain region affected by the biomarker.

12. The method of claim 1, wherein the description of biomarker tests results, interpretive analysis, and weighted index are presented on a written report.

13. The method of claim 1, wherein the description of biomarker tests results, interpretive analysis, and weighted index are presented electronically.

14. The method of claim 1, further comprising providing references specific to the patient's biomarker test result.

15. The method of claim 1, wherein the biomarker test result indicates polymorphism, deletion, repetition, insertion, methylation, expression level, expression localization, activity, or metabolites of one or more gene, protein, or neurotransmitter.

16. The method of claim 1, wherein the biomarker provides information about the autonomic arousal system of the patient's brain.

17. The method of claim 16, wherein the biomarker is a marker of a gene, or a protein encoded or modulated by gene selected from the group consisting of: SERT, SLC6A4 (SERT), 5HT1a, ACE, NPY, FKBP5, and other genes associated with heightened amygdala function.

18. The method of claim 1, wherein the biomarker provides information about the emotional valence, attention, reward and executive brain functions of the patient.

19. The method of claim 18, wherein the biomarker is a marker of a gene, or a protein encoded or modulated by gene selected from the group consisting of: COMT, sigma receptors, SNAp25, MAO A, SLC6A3, DRD2, DBH.

20. The method of claim 1, wherein the biomarker provides information about the patient's cognition and memory.

21. The method of claim 20, wherein the biomarker is a marker of a gene, or a protein encoded or modulated by gene selected from the group consisting of: CACNA1C, GRIK, GRM3, SLC1A1, ANK3, BDNF.

22. A method of presenting patient-specific pharmacodynamics information relevant to the treatment of a neuropsychiatric disorder, the method comprising:
presenting a description of a biomarker test result specific to a patient for at least one biomarker related to each of: the patient's autonomic arousal system, the patient's emotional valence, attention, reward and executive brain functions, and the patient's cognition and memory; and
presenting an interpretive analysis of the neurophysiological significance of each biomarker test result for the patient, wherein the interpretive analysis comprises pharmacodynamics information.

23. The method of claim 22, wherein the neuropsychiatric disorder is depression.

24. The method of claim 22, wherein the neuropsychiatric disorders is selected from the group including: treatment resistant depression, bipolar depression, anxiety disorders, PTSD, dementia, autism, and ADHD.

25. The method of claim 22, further comprising presenting a weighted index of confidence level for all or part of each interpretive analysis.

26. The method of claim 22, wherein the interpretive analysis further comprises the physiological significance each of the biomarker test results.

27. The method of claim 22, wherein the interpretive analysis further comprises a description of published studies describing similar biomarker test results.

28. The method of claim 22, wherein presenting an interpretive analysis comprises indicating possible drug responses.

29. The method of claim 22, further comprising presenting a description of a biomarker test results for a pharmacokinetic biomarker.

30. The method of claim 22, further comprising presenting a visual representation of a brain region affected by each biomarker.

31. The method of claim 22, wherein the description of biomarker tests results and interpretive analysis are presented on a written report.

32. The method of claim 22, wherein the description of biomarker tests results and interpretive analysis are presented electronically.

33. The method of claim 22, wherein the biomarker test results indicate polymorphism, deletion, repetition, insertion, methylation, expression level, expression localization, activity, or metabolites of one or more gene, protein, or neurotransmitter.

34. The method of claim 22, wherein the biomarker related to the autonomic arousal system of the patient's brain is a marker of a gene, or a protein encoded or modulated by gene selected from the group consisting of: SERT, SLC6A4 (SERT), ACE, NPY, FKBP5, HTR1A.

35. The method of claim 22, wherein the biomarker related to the emotional valence, attention, reward and executive brain functions of the patient's patient is a marker of a gene, or a protein encoded or modulated by gene selected from the group consisting of: COMT, sigma receptors, SNAp25, MAO A, SLC6A3, DRD2, DBH.

36. The method of claim 22, wherein the biomarker related to the patient's cognition and memory is a marker of a gene, or a protein encoded or modulated by gene selected from the group consisting of: CACNA1C, SCN1A, GRIK, GRM3, GRIK4, SLC1A1, ANK3, BDNF.

37. A method of presenting patient-specific pharmacodynamics information relevant to the treatment of a neuropsychiatric disorder, the method comprising:
providing a patient identifier;
presenting a description of a plurality of biomarker test results specific to the patient;
presenting an interpretive analysis of the neurophysiological significance of each of the biomarker test result for the patient, wherein the interpretive analysis comprises pharmacodynamics information; and
presenting a visual representation of a brain region affected by each biomarker.

38. The method of claim 37, wherein the neuropsychiatric disorder is depression.

39. The method of claim 37, wherein the neuropsychiatric disorders is selected from the group including: treatment resistant depression, bipolar depression, anxiety disorders, PTSD, dementia, autism, and ADHD.

40. The method of claim 37, further comprising presenting a weighted index of confidence level for the interpretive analysis.

41. The method of claim 37, wherein presenting the description of biomarker test results comprises presenting at least one biomarker related to each of: the patient's autonomic arousal system, the patient's emotional valence, attention, reward and executive brain functions, and the patient's cognition and memory.

42. The method of claim 37, wherein the interpretive analysis further comprises the physiological significance each of the biomarker test results.

43. The method of claim 37, wherein the interpretive analysis further comprises a description of published studies describing similar biomarker test results.

44. The method of claim 37, wherein presenting an interpretive analysis comprises indicating possible drug responses.

45. The method of claim 37, further comprising presenting a description of a biomarker test results for a pharmacokinetic biomarker.

46. The method of claim 45, wherein presenting a description of a plurality of biomarker test results comprises presenting a description of a biomarker test results for one or more markers of genes and/or proteins encoded or modulated by genes selected from the group consisting of: 2D6, 2C19, 3A4, ABCB1, 5HT2C, MTHF, MCR4, IDE.

47. The method of claim 37, wherein the description of biomarker tests results and interpretive analysis are presented on a written report.

48. The method of claim 37, wherein the description of biomarker tests results and interpretive analysis are presented electronically.

49. The method of claim 37, wherein the biomarker test results indicate polymorphism, deletion, repetition, insertion, methylation, expression level, expression localization, activity, or metabolites of one or more gene, protein, or neurotransmitter.

50. The method of claim 37, wherein presenting a description of a plurality of biomarker test results comprises presenting a description of a plurality of biomarker test results for two or more markers of genes and/or proteins encoded or modulated by genes selected from the group consisting of: SERT, SLC6A4 (SERT), SLC6A2, SLC6A3, 5HT2C, 5HT1a, ACE, NPS, TPH2, FKBP5, HTR1A, COMT, sigma receptors, SNAp25, MAO A, SLC6A3, DRD2, MTHFR, DBH, ANKK1, BDNF, CACNA1C, SCN1A, GRIK, GRM3, GRIK4, SLC1A1, ANK3.

51. The method of claim 37, wherein presenting a description of a plurality of biomarker test results comprises presenting a description of a plurality of biomarker test results for three or more markers of genes and/or proteins encoded or modulated by genes selected from the group consisting of: SERT, SLC6A4 (SERT), SLC6A2, SLC6A3, 5HT2C, FKBP5, FKBP5, HTR1A, COMT, sigma receptors, SLC6A3, DRD2, MTHFR, CACNA1C, SCN1A, GRIK, GRM3, GRIK4, DRD2, ANKK1, BDNF.

52. An article of manufacture comprising an interpretive neuropsychiatric report of patient-specific pharmacodynamics information relevant to the treatment of a neuropsychiatric disorder, the article of manufacture comprising:
a patient identifier;
a description of a biomarker test result specific to the patient;
an interpretive analysis of the neurophysiological significance of the biomarker test result for the patient, wherein the interpretive analysis comprises pharmacodynamics information; and
a weighted index of confidence level for the interpretive analysis.

53. The article of manufacture of claim 52, further comprising a plurality of descriptions of biomarker test results specific to the patient for a plurality of biomarkers.

54. The article of manufacture of claim 53, further comprising interpretive analyses of the neurophysiological significance of each of the biomarker test results for the patient.

55. The article of manufacture of claim 52, wherein the interpretive analysis further comprises a description of the physiological significance of the biomarker test result for the patient.

56. The article of manufacture of claim 52, wherein the interpretive analysis further comprises a description of published studies describing similar biomarker test results.

57. The article of manufacture of claim 52, wherein the weighted index of confidence level comprises an alphanumerical index of all or a portion of the interpretive analysis with a score indicating the type and/or number of studies supporting the interpretive analysis.

58. The article of manufacture of claim 52, wherein the interpretive analysis comprises an indicator of possible drug responses.

59. The article of manufacture of claim 52, further comprising a description of a biomarker test results for a pharmacokinetic biomarker.

60. The article of manufacture of claim 52, further comprising a visual representation of a brain region affected by the biomarker.

61. The article of manufacture of claim 52, wherein the description of biomarker tests results, interpretive analysis, and weighted index are presented on a written report.

62. The article of manufacture of claim 52, wherein the description of biomarker tests results, interpretive analysis, and weighted index are presented electronically.

63. The article of manufacture of claim 52, further comprising a list of references specific to the patient's biomarker test result.

64. The article of manufacture of claim 52, wherein the biomarker test result indicates polymorphism, deletion, repetition, insertion, methylation, expression level, expression localization, activity, or metabolites of one or more gene, protein, or neurotransmitter.

65. The article of manufacture of claim 52, wherein the biomarker provides information about the autonomic arousal system of the patient's brain.

66. The article of manufacture of claim 65, wherein the biomarker is a marker of a gene, or a protein encoded or modulated by gene selected from the group consisting of: SERT, SLC6A4 (SERT), 5HT1a, ACE, NPY, FKBP5, HTR1A.

67. The article of manufacture of claim 52, wherein the biomarker provides information about the emotional valence, attention, reward and executive brain functions of the patient.

68. The article of manufacture of claim 67, wherein the biomarker is a marker of a gene, or a protein encoded or modulated by gene selected from the group consisting of: COMT, sigma receptors, SNAp25, MAO A, SLC6A3, DRD2, MTHFR, DBH.

69. The article of manufacture of claim 52, wherein the biomarker provides information about the patient's cognition and memory.

70. The article of manufacture of claim 69, wherein the biomarker is a marker of a gene, or a protein encoded or modulated by gene selected from the group consisting of: CACNA1C, SCN1A, GRIK, GRM3, GRIK4, SLC1A1, ANK3, BDNF.

71. An article of manufacture comprising an interpretive neuropsychiatric report of patient-specific pharmacodynamics information relevant to the treatment of depression, the article of manufacture comprising:
a description of a biomarker test result specific to a patient for at least one biomarker related to each of: the patient's autonomic arousal system, the patient's emotional valence, attention, reward and executive brain functions, and the patient's cognition and memory; and
an interpretive analysis of the neurophysiological significance of each biomarker test result for the patient, wherein the interpretive analysis comprises pharmacodynamics information.

72. The article of manufacture of claim 71, further comprising a weighted index of confidence level for all or part of each interpretive analysis.

73. The article of manufacture of claim 71, wherein the interpretive analysis further comprises the physiological significance each of the biomarker test results.

74. The article of manufacture of claim 71, wherein the interpretive analysis further comprises a description of published studies describing similar biomarker test results.

75. The article of manufacture of claim 71, wherein the interpretive analysis indicates possible drug responses.

76. The article of manufacture of claim 71, further comprising a description of a biomarker test results for a pharmacokinetic biomarker.

77. The article of manufacture of claim 71, further comprising a visual representation of a brain region affected by each biomarker.

78. The article of manufacture of claim 71, wherein the description of biomarker tests results and interpretive analysis are presented on a written report.

79. The article of manufacture of claim 71, wherein the description of biomarker tests results and interpretive analysis are presented electronically.

80. The article of manufacture of claim 71, wherein the biomarker test results indicate polymorphism, deletion, repetition, insertion, methylation, expression level, expression localization, activity, or metabolites of one or more gene, protein, or neurotransmitter.

81. The article of manufacture of claim 71, wherein the biomarker related to the autonomic arousal system of the patient's brain is a marker of a gene, or a protein encoded or modulated by gene selected from the group consisting of: SERT, SLC6A4 (SERT), 5HT1a, ACE, NPY, FKBP5, HTR1A.

82. The article of manufacture of claim 71, wherein the biomarker related to the patient's emotional valence, attention, reward and executive brain functions is a marker of a gene, or a protein encoded or modulated by gene selected from the group consisting of: COMT, sigma receptors, SNAp25, MAO A, SLC6A3, DRD2, MTHFR, DBH.

83. The article of manufacture of claim 71, wherein the biomarker related to the patient's cognition and memory is a marker of a gene, or a protein encoded or modulated by gene selected from the group consisting of: CACNA1C, SCN1A, GRIK, GRM3, GRIK4, SLC1A1, ANK3, BDNF.

84. The article of manufacture of claim 71, further comprising a description of a biomarker test result for a pharmacokinetic biomarker.

85. The method of claim 84, wherein the description of a biomarker test result for a pharmacokinetic biomarker comprises presenting a description of a biomarker test results for one or more markers of genes and/or proteins encoded or modulated by genes selected from the group consisting of: 2D6, 2C19, 3A4, ABCB1, 5HT2C, MTHF, MCR4, IDE.

86. An article of manufacture comprising an interpretive neuropsychiatric report of patient-specific pharmacodynamics information relevant to the treatment of a neuropsychiatric disorder, the article of manufacture comprising:
a patient identifier;
a description of a plurality of biomarker test results specific to the patient;
an interpretive analysis of the neurophysiological significance of each of the biomarker test result for the patient, wherein the interpretive analysis comprises pharmacodynamics information; and
a visual representation of a brain region affected by each biomarker.

87. The article of manufacture of claim 86, wherein the interpretive neuropsychiatric report of patient-specific pharmacodynamics information relevant to the treatment of depression.

88. The article of manufacture of claim 86, wherein the interpretive neuropsychiatric report of patient-specific pharmacodynamics information relevant to the treatment of: treatment resistant depression, bipolar depression, anxiety disorders, dementia, autism, and ADHD.

89. The article of manufacture of claim 86, further comprising a weighted index of confidence level for the interpretive analysis.

90. The article of manufacture of claim 86, wherein the description of biomarker test results comprises a description of at least one biomarker related to each of: the patient's autonomic arousal system, the patient's emotional valence, attention, reward and executive brain functions, and the patient's cognition and memory.

91. The article of manufacture of claim 86, wherein the interpretive analysis further comprises the physiological significance each of the biomarker test results.

92. The article of manufacture of claim 86, wherein the interpretive analysis further comprises a description of published studies describing similar biomarker test results.

93. The article of manufacture of claim 86, wherein the interpretive analysis comprises a description of possible drug responses.

94. The article of manufacture of claim 86, further comprising a description of a biomarker test results for a pharmacokinetic biomarker.

95. The article of manufacture of claim 86, wherein the description of biomarker tests results and interpretive analysis are presented on a written report.

96. The article of manufacture of claim 86, wherein the description of biomarker tests results and interpretive analysis are presented electronically.

97. The article of manufacture of claim 86, wherein the biomarker test results indicate polymorphism, deletion, repetition, insertion, methylation, expression level, expression localization, activity, or metabolites of one or more gene, protein, or neurotransmitter.

98. The article of manufacture of claim 86, wherein the description of a plurality of biomarker test results comprises presenting a description of a plurality of biomarker test results for two or more markers of genes and/or proteins encoded or modulated by genes selected from the group consisting of: SERT, SLC6A4 (SERT), 5HT1a, ACE, NPY, FKBP5, HTR1A, COMT, sigma receptors, SNAp25, MAO A, SLC6A3, DRD2, MTHFR, DBH, ANKK1, BDNF, CACNA1C, SCN1A, GRIK, GRM3, GRIK4, SLC1A1, ANK3.

99. The article of manufacture of claim 86, wherein the description of a plurality of biomarker test results comprises presenting a description of a plurality of biomarker test results for three or more markers of genes and/or proteins encoded or modulated by genes selected from the group consisting of: SERT, SLC6A4 (SERT), 5HT1a, ACE, NPY, FKBP5, HTR1A, COMT, sigma receptors, SNAp25, MAO A, SLC6A3, DRD2, MTHFR, DBH, ANKK1, BDNF, CACNA1C, SCN1A, GRIK, GRM3, GRIK4, SLC1A1, ANK3.

100. A method of diagnosing a neuropsychiatric disorder based on patient-specific pharmacodynamics information, the method comprising:
sampling a patient;
testing the sample for at least one biomarker related to each of: the patient's autonomic arousal system, the patient's emotional valence, attention, reward and executive brain functions, and the patient's cognition and memory;
providing a report including the results of the biomarker test, an interpretive analysis of the neurophysiological significance of each biomarker test result, and a weighted index of confidence level for the interpretive analysis.

101. A system for generating a patient-specific pharmacodynamics report relevant to the treatment of a neuropsychiatric disorder, the system comprising:
an input module configured to receive at least one biomarker test result specific to a patient for each of: the patient's autonomic arousal system, the patient's emotional valence, attention, reward and executive brain functions, and the patient's cognition and memory;
an analysis module coupled to the input module and configured to generate an interpretive report from the plurality of biomarker test results, wherein the analysis module generates interpretive comment for each biomarker based on the test result.

102. A system for diagnosing or guiding a therapeutic treatment of a neuropsychiatric disorder, the system comprising:
an assay for determining the status of at least one biomarker related to each of: the patient's autonomic arousal system, the patient's emotional valence, attention, reward and executive brain functions, and the patient's cognition and memory; and
a report including the status of the biomarkers determined, an interpretive analysis of the neurophysiological significance of each biomarker's status, and a weighted index of confidence level for the interpretive analysis.
